(19) United States
(12) Patent Application Publication
(10) Pub. No.: US 2014/0037548 A1
(43) Pub. Date: Feb. 6, 2014

(54) Treatment and prevention of diffuse parenchymal lung disease by selective active-site mTOR inhibitors

(71) Applicants: Glenn D. Rosen, Los Altos, CA (US); Wen-Teh Chang, San Jose, CA (US)

(72) Inventors: Glenn D. Rosen, Los Altos, CA (US); Wen-Teh Chang, San Jose, CA (US)

(73) Assignee: The Board of Trustees of the Leland Standford Junior University, Palo Alto, CA (US)

(21) Appl. No.: 13/867,123

(22) Filed: Apr. 21, 2013

Related U.S. Application Data
(63) Continuation-in-part of application No. 13/171,441, filed on Jun. 28, 2011, now abandoned.
(60) Provisional application No. 61/398,622, filed on Jun. 28, 2010.
(60) Provisional application No. 61/459,317, filed on Dec. 8, 2010.

(51) Int. Cl. A61K 31/5377; A61K 49/00; A61K 31/519

(52) U.S. Cl. CPC A61K 31/5377; A61K 31/519; A61K 49/00
USPC 424/9.2; 514/262.1; 514/232.5

(57) ABSTRACT

Provided herein are methods for treating or preventing pulmonary fibrosis in subjects suffering from diffuse parenchymal lung diseases using selective active-site mTOR inhibitors.

### RELATED APPLICATION

This application is a continuation-in-part application of U.S. patent application Ser. No. 13/171,441, filed Jun. 28, 2011, entitled “Treatment and Prevention of diffuse parenchymal Lung Disease by selective active-site mTOR Inhibitors”, which claims priority and other benefits from U.S. Provisional Patent application Ser. No. 61/398,622, filed Jun. 28, 2010, entitled “Inhibition of Pulmonary Fibrosis by Dual mTOR Inhibitors”, and Ser. No. 61/459,317, filed Dec. 8, 2010, entitled “Treatment and Prevention of Pulmonary Fibrosis by Selective Active-Site mTOR Inhibitors”. Their entire content is specifically incorporated herein by reference.

### TECHNICAL FIELD OF THE INVENTION

The invention relates generally to the treatment of lung diseases, and more particularly, to the treatment of diffuse parenchymal lung diseases using selective active-site inhibitors of the mammalian target of rapamycin (mTOR).

### BACKGROUND

Diffuse parenchymal lung diseases (DPLDs), also known as interstitial lung diseases, are devastating diseases of often unknown cause, usually with poor prognosis and limited treatment options.

Idiopathic pulmonary fibrosis (IPF) is a chronic and particularly progressive form of diffuse parenchymal lung disease that is characterized by an accumulation of scar tissue in the lung's interstitium and for which currently no effective pharmacological treatment is available in the United States, except for pirfenidone which is approved in several countries and continents outside of the United States, such as in Europe, Australia, India, South Korea and Japan. Idiopathic pulmonary fibrosis occurs primarily in subjects over the age of 50, who have a median survival time of 4-5 years, before they succumb to respiratory failure.

There is an urgent and unmet need to identify agents and methods for the effective treatment of idiopathic pulmonary fibrosis and other fibrotic diseases.

### SUMMARY

The present invention provides methods for treating diffuse parenchymal lung diseases and for blocking their pathogenesis.

A first embodiment is a method for treating or preventing pulmonary fibrosis in an subject suffering from a diffuse parenchymal lung disease and administering an amount of a pharmaceutical composition comprising a selective active-site mTOR inhibitor to said subject effective to treat or prevent pulmonary fibrosis in said subject. Another embodiment is a method for determining treatment efficacy of an subject's treatment with an active-site mTOR inhibitor by assessing said subject's lung function over the course of the treatment.

A further embodiment is a method for inhibiting expression of α-SMA or collagen or fibronectin or secreted protein acidic and rich in cysteine (SPARC) in a pulmonary fibroblast comprising administering an amount of a selective active-site mTOR inhibitor to a pulmonary fibroblast in an subject suffering from a diffuse parenchymal lung disease effective to inhibit expression of α-SMA or collagen or fibronectin or SPARC in said pulmonary fibroblast of said subject.

In further embodiments said diffuse parenchymal lung disease is a diffuse parenchymal lung disease of environmental cause or a collagen vascular disease, such as scleroderma or rheumatoid arthritis, or an idiopathic interstitial pneumonia or sarcoidosis.

In other embodiments, said idiopathic interstitial pneumonia is idiopathic pulmonary fibrosis (IPF) or nonspecific interstitial pneumonia (NSIP). In certain embodiments said selective active-site mTOR inhibitor is a pyrazolopyrimidine.

In some embodiments said selective active-site mTOR inhibitor is Torin 1, PP242, INK128, Ku-0063794, or WAY-600.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1. Diffuse parenchymal lung diseases (DPLDs) encompass a collection of disorders. Am J Respir Crit. Care Med., 2002, 165, 277-304.

FIG. 2. The diagnostic method in diffuse pulmonary lung diseases (DPLDs) is based on clinical, radiological, and pathological criteria. Am J Respir Crit. Care Med., 2002, 165, 277-304.

FIG. 3. mTORC1 and mTORC2 are distinguished by different compositions, substrates, and functions. J Biol. Chem., 2010, 285, 14071-7.

FIG. 4. In vitro IC₅₀ Values for PP242 and PP30 determined in the presence of 10 μM ATP. PLoS Biol., 2009, 7, e38.

FIG. 5. Schematic of signaling through SPARC in IPF fibroblasts. J Biol. Chem., 2010, 285, 8196-206.

FIG. 6. Working model of dual mTOR signaling in pulmonary fibrogenesis. Cf Wang, S. et al., Am J Physiol Renal Physiol., 2010, 298, F142-9. Following TGF-β stimulation in pulmonary fibroblasts, the noncanonical and canonical (through Smad2/Smad3) TGF-β pathways closely collaborate. The pathways induce transition of fibroblasts into myofibroblasts and the expression in these fibroblasts and myo-fibroblasts of stromal proteins, including α-smooth muscle actin (α-SMA) and multiple extracellular matrix proteins, such as collagen and fibronectin. According to Wang et al., supra, the noncanonical pathway also induces fibroblast proliferation through the Akt-mTORC1-S6K branch.

FIG. 7 shows the structures of the selective active-site mTOR inhibitors Torin 1, WAY-600, Ku-0063794, PP242 and INK128.

FIG. 8. Panel A illustrates that active site or dual mTOR inhibitors, PP242 or Ku-0063794 (which block both mTORC1 and mTORC2) are more effective than rapamycin (which primarily blocks mTORC1) in suppressing expression of scar-forming extracellular matrix proteins, type I collagen and secreted protein acidic and rich in cysteine (SPARC) from fibroblasts, which are derived from subjects with idiopathic pulmonary fibrosis (IPF). Active-site mTOR inhibitors PP242 and Ku-0063794, but not rapamycin, block phosphorylation of Akt at Ser473, a target of mTORC2 (Panel B).

FIG. 9 illustrates that transforming growth factor β (TGF-β), a major mediator of fibrosis in IPF, activates Akt (Serine 473 and Threonine 308) in normal lung fibroblasts, both of which are blocked by active-site mTOR inhibitors PP242 or Ku-0063794, but not by rapamycin. PP242 or Ku-0063794 is more effective than rapamycin in blocking induction by TGF-β of type I collagen, α-smooth muscle actin (α-SMA) and fibronectin, all proteins made by activated fibroblasts, which promote scar formation in IPF. Also, PP242 proved more effective than rapamycin or Ku-0063794 in blocking induction by TGF-β of plasminogen activator inhibitor 1 (PAI-1). As seen in FIG. 2D, none of these inhibitors affect the Smad pathway, which is the major TGF-β-regulated pathway.

FIG. 10 illustrates that turning off expression of Rictor, a component of the mTORC2 pathway, which when inhibited blocks mTORC2 but not mTORC1, shows similar repressive effects on these pro-fibrotic proteins to PP242 or Ku-0063794 in IPF fibroblasts and TGF-β-stimulated control fibroblasts. It is illustrated that a specific Akt inhibitor shows similar effects but not identical, suggesting that activation of Akt by mTORC2 mediates expression of some pro-fibrotic proteins by IPF fibroblasts and control TGF-β-stimulated fibroblasts.

FIG. 11 illustrates that PP242 significantly inhibited fibrosis in the bleomycin murine model of lung injury and fibrosis, which is a well-accepted animal model of lung injury and fibrosis. PP242 significantly inhibits expression of important matrix regulated proteins, which are upregulated by bleomycin and known targets of TGF-β, such as SPARC, metastasin (MTS-1/S100A4), and matrix metalloproteinase 7 (MMP-7). Quantitation of hydroxyproline content at day 14 in the right lung of saline treated mice, mice treated with Bleomycin plus vehicle and mice treated with Bleomycin plus pp 242 one day prior to the administration of Bleomycin. Results are the average of three independent experiments with mean±SD.

FIG. 12 illustrates that PP242 or repression of Rictor inhibited migration of control or IPF fibroblasts.

FIG. 13A shows the structures of dual (selective active-site) mTOR inhibitors PP242 and INK128 (MLN 0128). INK 128 is a second generation dual mTOR inhibitor, which is structurally similar to PP242, but about 10 times more potent than PP242.

FIG. 13B. mTOR ATP-competitive inhibitors, but not rapamycin, suppress TGF-beta induced expression of markers for activated fibroblasts. IPF fibroblasts were serum-starved for 24 hours prior to the treatment of TGF-beta (2 ng/ml) for over night in the presence or absence of indicated inhibitors (INK128 at 200 nM, PP242 at 2 uM, and Rapamycin at 50 nM). Total cell lysates were prepared and equal amount of proteins were analyzed by Western blot analysis with specific antibody against each protein. Alpha-tubulin was used as a loading control. Three separate experiments were performed and the represented results were shown here.

FIG. 14A. mTOR ATP-competitive inhibitors suppress TGF-beta induced Akt phosphorylation. IPF fibroblasts were treated as in FIG. 13B, except the treatment of TGF-beta was 60 minutes. Western blot analysis was applied to determine the phosphorylation levels of specific motifs of Akt proteins. Total Akt protein was used as a loading control.

FIG. 14B. mTOR ATP-competitive inhibitors, but not rapamycin, suppress TGF-beta induced Akt phosphorylation. mTOR inhibitors efficiently suppress S6 kinase activity following TGF-beta activation. IPF fibroblasts were serum-starved for 24 hours prior to the treatment of TGF-beta (2 ng/ml) for 6 hours in the presence or absence of indicated inhibitors (INK128 at 200 nM, PP242 at 2 uM, Rapamycin at 50 nM) for 6 hours. Total cell lysates were prepared and equal amount of proteins were analyzed by Western blot analysis against phosphorylated S6 proteins, as the substrates of S6 kinase.

FIG. 14C. mTOR inhibitors have no effect on TGF-beta induced Smad activation. IPF fibroblasts were treated as in (B), except the treatment of TGF-beta was 10 minutes. Western blot analysis was applied to determine the phosphorylation levels of Smad2 proteins. Total Smad2/3 protein was used as a loading control. Three separate experiments were performed for each assay and the represented results were shown here.

FIG. 15. Rictor but not Raptor regulates Akt phosphorylation. In (A) IPF fibroblasts were infected with lentivirus-derived shRNA against raptor or rictor, or control (scramble). Western blot followed with the indicated antibodies. α-tubulin was used as a loading control. (B) Serum-starved IPF fibroblasts were treated with TGF-β for 60 minutes followed by analysis of Akt phosphorylation by Western blot analysis. Total Akt was used as a loading control. (C). IPF fibroblasts were treated overnight with TGF-β followed by analysis of matrix-regulatory proteins by Western blot analysis. α-tubulin was used as a loading control. Three separate experiments were performed and representative results are shown.

FIG. 16. INK128 inhibits bleomycin-induced injury and fibrosis in the bleomycin treatment model. INK128 treatment began at Day 7 following intratracheal bleomycin administration. Mice were harvested at Day 21 for H&E staining (upper panel) or analysis of collagen with picosirius red immunohistochemistry (lower panel).

FIG. 17A. In the bleomycin prevention model, INK128 protects against bleomcyin injury and fibrosis. Quantitation of hydroxyproline content at Day 14 in the right lung of saline treated mice, mice treated with Bleomycin plus vehicle and mice treated with Bleomycin plus INK128 one day prior to the administration of Bleomycin.

FIG. 17B. Bleomycin treatment model. Mice were treated with INK128 beginning 7 days after bleomycin followed by harvest of right lung at Day 21 for analysis of hydroxyproline content

FIG. 17C. Mouse Left Lung Ashcroft Score (Prevention Experiment—Day 14). The fibrotic changes of mice left lungs were assessed by the Ashcroft scale. The paraffin sections of lung were stained with H&E and 30 successive fields were evaluated by a pulmonary pathologist, and each field was assessed for the severity with a score from 0 (normal) to 8 (honeycombing). Each group is presented with the average score.

FIG. 17D. Ashcroft score in bleomycin treatment model. INK128 treatment began at Day 7 following intratracheal bleomycin administration and mice were sacrificed at Day 21.

FIG. 17E. Mice Right Lung mRNA Level. INK128 (0.75 mg/kg) was given one day prior to the administration of Bleomycin (1.0 U/kg) and daily for 14 days post Bleomycin. Mice were sacrificed on Day 14. RNA from lungs treated as shown above was analyzed by quantitative PCR. Relative expression is normalized to the saline group and average fold change denotes the mean±SD.

FIG. 18. INK128 blocks TGF-β-mediated attenuation of lung epithelial cell viability.

(A) the Transwell culture protocol using IPF fibroblasts and A549 cells or (B) RLE-6TN cells is described in detail in Methods, which was followed by analysis of A549 or RLE-6TN viability by Alamar Blue.

FIG. 19. Downregulation of SPARC in A549 (A) or RLE-6Tn (B) cells or downregulation of Rictor in A549 (C) or RLE-6TN (D) cells by RNA interference in TGF-β-treated IPF fibroblasts was followed by an Alamar Blue assay of A549 or RLE-6TN cells. Data is expressed as mean+/−standard error of three independent experiments.

FIG. 20. H₂O₂ peroxide release from IPF fibroblasts is mediated by SPARC and mTORC2. (A) IPF fibroblasts were treated for 16 h with TGF-β alone or in combination with INK128 (0.2 μM) followed by measurement of H₂O₂, as described previously (Zhou, M., Diwu, Z., Panchuk-Voloshina, N., and Haugland, R. P. 1997. Analytical biochemistry 253:162-168). (B) SPARC or Rictor (C) was downregulated by RNA interference in TGF-β-treated IPF fibroblasts followed by measurement of H₂O₂. Data is expressed as mean+/−standard error of three independent experiments.

### DEFINITIONS

The practice of the present invention may employ conventional techniques of chemistry, molecular biology, recombinant DNA, genetics, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002); Paul Singleton and Diana Sainsbury, Dictionary of Microbiology and Molecular Biology (3d ed. revised, John Wiley & Sons, Chichester, England, 2006). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “drug” means, as used herein, a molecule intended for use in the diagnosis, or prevention of a disease.

The term “subject”, as used herein, refers to a mammal, including mouse, rabbit, dog, cat, guinea pig, goat, cow, horse, pig, sheep, monkey, primate, ape, and, preferably, a human.

The terms “selective active-site mTOR inhibitor”, “active-site mTOR inhibitor” and “dual mTOR inhibitor” are used interchangeably herein and represent inhibitors of both mTORC1 and mTORC2, whereby such inhibitors inhibit at least mTOR Complex 2 (mTORC2) and may also inhibit mTOR Complex 1 (mTORC1). An active-site mTOR inhibitor, thus, does not include agents that only inhibit mTORC1, such as rapamycin and rapamycin analogs.

The term “pharmaceutical composition”, as used herein, refers to a mixture of at least one active-site mTOR inhibitor with chemical components such as diluents or carriers that do not cause unacceptable adverse side effects and that do not prevent the active-site mTOR inhibitor(s) from exerting a therapeutic effect. A pharmaceutical composition serves to facilitate the administration of the active-site mTOR inhibitor (s).

Routes of administration of active-site mTOR inhibitors or pharmaceutical compositions containing active-site mTOR inhibitors may include, but are not limited to, oral or topical administration and intramuscular, subcutaneous, intravenous or intraperitoneal injections. The active-site mTOR inhibitors or pharmaceutical compositions containing active-site mTOR inhibitors may also be administered locally via an injection or in a targeted delivery system.

A therapeutic effect may include, directly or indirectly, the reduction of pulmonary interstitial fibrosis and the reduction of pulmonary interstitial inflammation. A therapeutic effect may also include, directly or indirectly, the arrest, reduction, or elimination of pulmonary interstitial fibrogenesis or extracellular matrix deposition. Furthermore, a therapeutic effect may include, directly or indirectly, the prevention or reduction of the expression of stromal genes constitutively expressed in idiopathic pulmonary fibrosis fibroblasts including, but not limited to fibronectin (FN), type-1-collagen, alpha smooth muscle actin (α-SMA), secreted protein acidic and rich in cysteine (SPARC) and plasminogen activator inhibitor 1 (PAI-1). Furthermore, a therapeutic effect may also include, directly or indirectly, the prevention or reduction of the expression of transforming growth factor beta (TGF-β).

The term “therapeutically effective amount” of an active-site mTOR inhibitor is an amount that is sufficient to provide a therapeutic effect in a subject. Naturally, dosage levels of the particular active-site mTOR inhibitor employed to provide a therapeutically effective amount may vary in dependence of the type of injury, the age, the weight, the gender, the medical condition of the subject, the severity of the condition, the route of administration, and the particular active-site mTOR inhibitor employed. Therapeutically effective amounts of an active-site mTOR inhibitor, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The therapeutic efficacy of treating a subject with an active-site mTOR inhibitor can be determined by repeatedly assessing said subject's lung function over the course of the treatment. Spirometry is the most common method to assess lung function, specifically measuring the amount (volume) and speed (flow) of air that can be inhaled and exhaled. An increase in lung function over time, in comparison to prior lung function tests, indicates therapeutic efficacy of treatment with an active-site mTOR inhibitor. Similarly, unchanged lung function over time, thus indicating an arrest in worsening of lung function, in comparison to prior lung function tests, can also indicate therapeutic efficacy of treatment with an active-site mTOR inhibitor. Accordingly, a decrease in lung function over time, in comparison to prior lung function tests, can indicate a lack of therapeutic efficacy of treatment with an active-site mTOR inhibitor.

Treatment with active-site mTOR inhibitors is carried out with a regular dosing regimen or schedule, e.g. daily, weekly, monthly and so forth, in the frequency that is sufficient to increase lung function over time, as one of the measures for treatment success. Dosing can occur once daily, twice daily, three times or more often daily; once, twice or three times of more often every other day; once, twice or three times of more often for several, consecutive days, weeks or months.

### DETAILED DESCRIPTION

The present invention provides methods for treating diffuse parenchymal lung diseases and for blocking their pathogenesis.

### Diffuse Parenchymal Lung Diseases

Diffuse Parenchymal Lung Diseases.

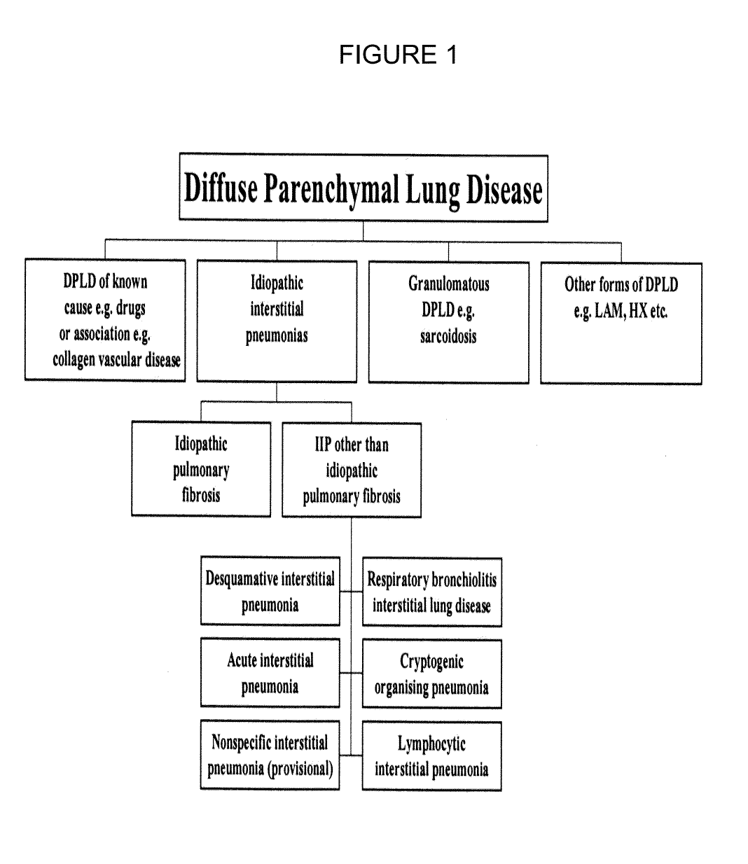

Diffuse parenchymal lung diseases (DPLDs), also known as interstitial lung diseases, encompass pulmonary disorders that affect the interstitium, i.e. the tissue and the space surrounding the air sacs of the lung. Such diffuse parenchymal lung diseases include disorders of known causes (collagen vascular disease, environmental or drug related) as well as disorders of unknown cause (FIG. 1).

Disorders of known causes include collagen vascular diseases and disorders that are precipitated through environmental factors or drug ingestion. Environmental factors that can trigger such disorders can be inhalation of smoke; inhalation or ingestion of inorganic dust particles such as from asbestos, silica, coal, aluminum; inhalation or ingestion of pesticides, irritant gases and fumes, fungal spores, bacterial spores and so forth.

The latter include idiopathic interstitial pneumonias (IIPs), granulomatous lung disorders (e.g., sarcoidosis), and other forms of interstitial lung disease (ILD) including lymphangioleiomyomatosis (LAM), pulmonary Langerhans' cell histiocytosis/histiocytosis X (HX), and eosinophilic pneumonia. The most important distinction among the idiopathic interstitial pneumonias is that between idiopathic pulmonary fibrosis and the other interstitial pneumonias (IPs), which include nonspecific interstitial pneumonia, desquamative interstitial pneumonia, respiratory bronchiolitis-associated interstitial lung disease, acute interstitial pneumonia, cryptogenic organizing pneumonia, and lymphocytic interstitial pneumonia.

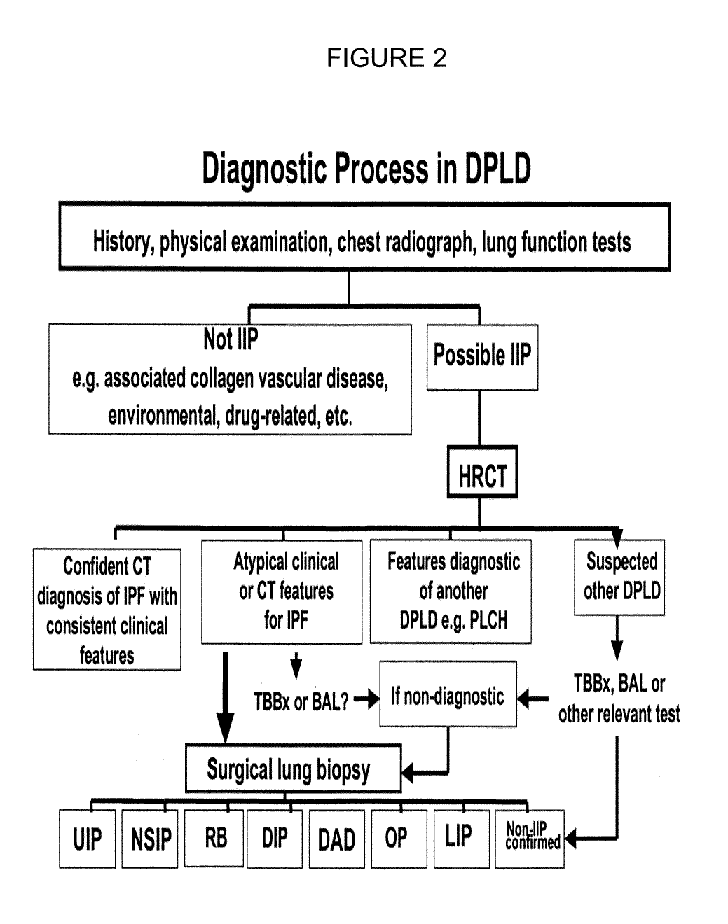

Referring to FIG. 2, the diagnostic method in diffuse pulmonary lung diseases (DPLDs) begins with a clinical evaluation that includes a history, physical examination, chest radiograph, and lung function tests. On the basis of this information, the subjects may be divided into two groups: cases that do not represent idiopathic interstitial pneumonia (IIP), owing to recognition of associated conditions or underlying exposures, and cases that could represent IIP. Subjects in the latter category typically receive a high-resolution computerized tomography (HRCT) scan. This generally results in four categories of subjects: (1) those with distinctive features that allow for a confident diagnosis of idiopathic pulmonary fibrosis (IPF)/usual interstitial pneumonia (UIP) in the appropriate clinical setting, (2) those with atypical clinical or CT features for IPF, (3) those with features diagnostic of another DPLD such as pulmonary Langerhans' cell histiocytosis (PLCH), and (4) those with suspected other forms of DPLD. Although many subjects will go directly to surgical lung biopsy, some subjects may undergo transbronchial biopsy (TBBx) or bronchoalveolar lavage (BAL). If these findings are nondiagnostic a surgical lung biopsy may be necessary to separate the various IIPs from non-IIP DPLD.

In general, most diffuse pulmonary lung disease is characterized by particular microscopic patterns of inflammation and fibrosis. Fibrosis is characterized by an increased amount and abnormal structure of the connective tissue. Inflammation is characterized by excessive inflammatory cells.

Idiopathic Interstitial Pneumonia.

Referring, again, to FIG. 1, the idiopathic interstitial pneumonias (IIPs) are a group of diffuse parenchymal lung diseases (DPLDs). The IIPs are a heterogeneous group of normeoplastic disorders resulting from damage to the lung parenchyma by varying patterns of inflammation and fibrosis. The interstitium includes the space between the epithelial and endothelial basement membranes and it is the primary site of injury in the IIPs. However, these disorders frequently affect not only the interstitium, but also the airspaces, peripheral airways, and vessels along with their respective epithelial and endothelial linings.

Idiopathic indicates unknown cause and interstitial pneumonia refers to involvement of the lung parenchyma by varying combinations of fibrosis and inflammation. The idiopathic interstitial pneumonias include the entities of idiopathic pulmonary fibrosis (IPF), nonspecific interstitial pneumonia (NSIP), cryptogenic organizing pneumonia (COP), acute interstitial pneumonia (AIP), respiratory bronchiolitis-associated interstitial lung disease (RB-ILD), desquamative interstitial pneumonia (DIP), and lymphocytic interstitial pneumonia (LIP).

The current clinico-pathologic classification of the idiopathic interstitial pneumonias (IIPs) is summarized in Table 1. See Am J Respir Crit. Care Med., 2002, 165, 277-304; Strollo D. C., Am J Respir Cell Mol. Biol., 2003, 29(3 Suppl), S10-18.

TABLE 1
Classification of Idiopathic Interstitial Pneumonias

| Diagnosis | Radiology | Distribution | Pathology |
|---|---|---|---|
| IPF/UIP | Fibrosis, | Basilar, peripheral | Temporal |
| | honeycombing | | heterogeneity, |
| | | | fibroblastic foci, |
| | | | fibrotic and normal |
| | | | lung, microscopic |
| | | | honeycombing |
| NSIP | Ground glass | Basilar, peripheral | Diffuse interstitial |
| | opacity +/− | | inflammation +/− |
| | fibrosis | | fibrosis |
| COP | Ground glass | Patchy upper lungs, | Granulation tissue |
| | opacity, nodules, | small airways, | plugs in alveolar |
| | consolidation | alveolar | ducts and alveoli |
| AIP | Ground glass | Diffuse, random | Hyaline |
| | opacity, | | membranes, |
| | consolidation | | immature |
| | | | fibroblasts in |
| | | | alveolar spaces and |
| | | | interstitium to |
| | | | variable degree |
| RB-ILD | Bronchiectasis, | Upper lungs, | Respiratory |
| | ground glass | bronchocentric | bronchiolitis |
| | opacity | | surrounded by |
| | | | macrophages in |
| | | | alveoli |
| DIP | Ground glass | Basilar, peripheral, | Alveolar |
| | opacity, | alveolar | macarophages in |
| | consolidation | | air spaces diffusely |
| | | | in the biopsy |
| LIP | Ground glass | Patchy | Lymphoid |
| | opacity, nodules, | | hyperplasia |
| | cysts | | |

Idiopathic Pulmonary Fibrosis.

The terms usual interstitial pneumonia (UIP) and idiopathic pulmonary fibrosis (IPF) have become more narrowly defined since they were originally proposed several decades ago. The relationship between historically defined IPF, or cryptogenic fibrosing alveolitis (CFA), and UIP has been described in Am J Respir Crit. Care Med., 2000, 161, 646-664. According to the current definition, IPF is a distinctive type of chronic fibrosing interstitial pneumonia of unknown cause limited to the lungs and associated with a surgical lung biopsy showing a histologic pattern of UIP. Am J Respir Crit. Care Med., 2000, 161, 646-664.

A dysregulated wound healing response to lung epithelial injury, which leads to progressive interstitial fibrosis, is a hallmark of the disease. The fibrotic scar is generated, in large part, by activated fibroblasts, which are sometimes referred to as myofibroblasts, based on their expression of alpha smooth muscle actin (α-SMA). Lung fibroblasts transdifferentiate into myofibroblasts in response to TGF-β, which is released largely by injured type I or type II alveolar epithelial cells and activated macrophages in fibroblastic foci in IPF lung. These myofibroblasts produce type I and type III collagen, which compose the fibrotic interstitial scar, and other mesenchymal proteins such as secreted protein acidic and rich in cysteine (SPARC), plasminogen activator inhibitor 1 (PAI-1), and fibronectin (FN).

In the presence of a surgical biopsy showing a UIP pattern the diagnosis of IPF requires (1) exclusion of other known causes of interstitial lung disease including drug toxicities, environmental exposures, and collagen vascular diseases, (2) characteristic abnormalities on conventional chest radiographs or high-resolution computed tomography (HRCT) scans, and (3) abnormal pulmonary function studies showing restriction (reduced total lung capacity [TLC], or reduced vital capacity [VC] with a normal or increased FEV₁/FVC ratio) and/or impaired gas exchange [increased P(A-a)O₂ (alveolar-arterial pressure difference for O₂), decreased Pa_{O2} with rest or exercise, or decreased DL_{CO} (diffusing capacity of the lung for CO)].

The new American Thoracic Society (ATS)/European Respiratory Society (ERS)/Japanese Respiratory Society (JRS)/Latin American Thoracic Society (ALAT) guidelines for the diagnosis and management of IPF were published in the Am J Respir Crit. Care Med, 2011, 183, 788-824. However, lung biopsy may occasionally not be definitive. This may arise when there is histologic heterogeneity in different lobes of the lung in IPF. So after correlating all the clinical, radiological, and pathological information, the final diagnosis may still be IPF in a subject with typical clinical-radiological IPF, even though a lung biopsy shows a fibrosing NSIP pattern.

Clinical Features.

Onset of symptoms is usually gradual, with dyspnea the most prominent and disabling symptom. A nonproductive cough is usual and may be paroxysmal. It is often refractory to antitussive agents. The subject's age at onset is usually greater than 50 yr and IPF is slightly more common in males. Constitutional symptoms are unusual. Digital clubbing develops in 25 to 50% of subjects, and Velcro-type fine end-inspiratory crackles that are initially confined to the basal areas are found on chest auscultation. These progress gradually to involve the entire lung. Features of right heart failure and peripheral edema develop only in the late stages. Most subjects exhibit a restrictive pattern of ventilatory defect with a decrease in DL_{CO} and low resting Pa_{O2}, which falls on exercise. Pulmonary function or chest radiographs may be normal or near normal in the early phase of IPF. In smokers and ex-smokers with IPF, coexistent chronic obstructive pulmonary disease may result in relatively higher lung volumes compared with never-smoking subjects with IPF.

In most subjects, symptoms have been present for more than 6 month before presentation. The clinical course is invariably one of gradual deterioration. Median length of survival from time of diagnosis varies between 4-5 yrs. Occasionally, periods of rapid decline are recognized. These may represent accelerated disease, intercurrent viral infection with the development of organizing pneumonia, or diffuse alveolar damage. Improvement in lung physiology and radiologic abnormalities is rare.

Bronchoalveolar lavage fluid contains an excess of neutrophils, the proportions of which correspond to the extent of reticular change on HRCT. There may also be a mild or moderate increase in the percentage of eosinophils. BAL cell counts, although correlating with severity of disease, do not predict prognosis. When eosinophils represent more than 20% of the count, consideration should be given to an eosinophilic lung disease. Lymphocytosis is not a feature of UIP, and counts above 15% should alert to an alternative diagnosis such as NSIP, COP, hypersensitivity pneumonitis, sarcoidosis, or other granulomatous lung disease.

Radiologic Features.

The commonest chest radiographic abnormality in subjects with IPF is peripheral reticular opacity, most marked at the bases, and often associated with honeycombing and lower lobe volume loss. In subjects with associated upper lobe emphysema, the radiographic lung volumes may be normal or even increased. Chest radiographs may occasionally be normal in subjects with IPF.

UIP is characterized on CT by the presence of reticular opacities, often associated with traction bronchiectasis. Honeycombing is common. Ground glass opacities are uncommon, but ground glass attenuation, which is associated with areas of fibrosis, can often be seen. Architectural distortion, reflecting lung fibrosis, is often prominent. Lobar volume loss is seen with more advanced fibrosis. The distribution of UIP on CT is characteristically basal and peripheral, although often patchy. On serial scans in treated subjects, honeycomb cysts usually enlarge slowly over time.

Reticular abnormality on CT correlates with fibrosis on histopathologic examination. Honeycombing on CT correlates with honeycombing on biopsy. The CT pattern of UIP due to IPF can be indistinguishable from that found in UIP due to asbestosis and to collagen vascular disease. The presence of pleural plaques or diffuse pleural thickening helps to distinguish asbestosis from IPF. Subjects with chronic hypersensitivity pneumonitis, or with end-stage sarcoidosis, may uncommonly develop a CT pattern similar to that of UIP. Hypersensitivity pneumonitis should be considered if poorly defined fine micronodules are seen, or if there is sparing of the lung bases. Sarcoidosis should be suspected if the cysts are large, or if peribronchovascular nodules are present.

Histologic Features.

The key histologic features of the UIP pattern are architectural destruction, fibrosis often with honeycombing, scattered fibroblastic foci, patchy distribution and involvement of the periphery of the acinus or lobule. It has a heterogeneous appearance at low magnification, with alternating areas of normal lung, interstitial inflammation, fibrosis, and honeycomb change. The histological changes affect the peripheral subpleural parenchyma most severely. Interstitial inflammation is usually mild to moderate, patchy, and consists of an alveolar septal infiltrate of lymphocytes, plasma cells, and histiocytes associated with hyperplasia of Type II pneumocytes. The fibrotic zones show temporal heterogeneity with dense acellular collagen and scattered fibroblastic foci. Areas of honeycomb change are composed of cystic fibrotic airspaces, which are frequently lined by bronchiolar epithelium and filled with mucin. Smooth muscle hyperplasia is commonly seen in areas of fibrosis and honeycomb change. Areas of relatively normal lung should be present in surgical biopsy specimens in order to exclude the presence of active lesions of other interstitial disorders. Otherwise the UIP pattern may be difficult to recognize and a pathologist may only be able to diagnose “severe fibrosis with honeycomb change.” In some subjects with a UIP pattern on lung biopsy, specimens from a second or third lobe of lung may not fulfill the histologic criteria for UIP and suggest other patterns such as NSIP. However, in such a setting the default pathologic diagnosis is UIP.

Subjects who are biopsied during an accelerated phase of their illness may show a combination of UIP pattern and a variety of acute lesions. These include infection, prominent organizing pneumonia, diffuse alveolar damage, and capillaritis. If no cause can be determined this may represent “accelerated decline of IPF” or acute exacerbation of IPF. A pattern of interstitial inflammation and fibrosis nearly indistinguishable from that seen in UIP can occur in subjects with collagen vascular diseases, certain drug-induced lung diseases, chronic hypersensitivity pneumonitis, asbestosis, and familial IPF. There is no single histologic finding that has shown a consistent correlation with treatment response or prognosis in IPF.

The differential diagnosis of the IIPs must be approached in two ways: histologically and clinically. In interpreting lung biopsies, the pathologist must address the differential diagnosis on the basis of the histologic pattern. A search should be made for histologic clues to a potential cause such as asbestos bodies, infectious agents, or other exogenous agents. The clinician must address most of the etiologic possibilities and in most cases ultimately determines whether the process is idiopathic.

The histologic differential diagnosis of the UIP pattern includes the histologic patterns of the other IIPs including fibrosing NSIP, DIP, organizing pneumonia, chronic hypersensitivity pneumonitis, and diffuse alveolar damage. With the narrowing of the histologic definition of the UIP pattern there are only a few clinical conditions that may cause an identical histologic pattern. Lesions that can present histological features similar but not identical to UIP include asbestosis, collagen vascular disease, the fibrosing phase of hypersensitivity pneumonitis, radiation pneumonitis, and Hermansky-Pudlak syndrome.

Collagen Vascular Disease.

Collagen vascular or connective tissue disorders are a group of autoimmune diseases in which antibodies attack the body's own organs and systems. Among the many targets of these auto-antibodies is connective tissue, which is the supporting structure for all of the body's cells. An important component of connective tissue is the protein, collagen. Abnormalities in blood vessel structure and function are also typical, accounting for the term “collagen vascular diseases,” which is often used interchangeably with connective tissue disorders. These disorders typically feature inflammation and fibrosis in several organs and tissues. The joints are frequently involved, particularly in the most common of these conditions, rheumatoid arthritis. Thus, rheumatology is the primary medical subspecialty involved in the diagnosis and care of these subjects. Lung involvement may complicate the course of most of these conditions and sometimes can dominate the clinical picture.

The frequency and type of lung involvement in connective tissue disorders varies based on the underlying disease. Of all the connective tissue disorders, scleroderma, also known as systemic sclerosis, is most likely to affect the lungs. Pulmonary fibrosis occurs in over two thirds of scleroderma subjects, but pulmonary fibrosis also occurs in rheumatoid arthritis, mixed connective tissue disease, systemic lupus erythematosus, Sjögren's syndrome, and polymyositis/dermatomyositis.

Scleroderma.

Scleroderma, also known as systemic sclerosis, is a rheumatic and autoimmune disease characterized by skin thickening and effects on the vascular system, lung, kidney, gastrointestinal tract, and heart. The criteria for the classification of scleroderma are published as Arthritis Rheum 1980, 23, 581-90.

Rheumatoid Arthritis.

Rheumatoid arthritis (RA) is a rheumatic and autoimmune disease characterized by inflammation in the joints. The 1987 American College of Rheumatology criteria are used in the clinical diagnosis of rheumatoid arthritis, and to define rheumatoid arthritis in epidemiologic studies. Persons must meet four of seven ACR criteria; these criteria are based on clinical observation (e.g., number of joints affected), laboratory tests (e.g., positive rheumatoid factor), and radiographic examination (e.g., X-rays evidence of joint erosion). Arnett F C et al., Arthritis Rheum 1988, 31, 315-324.

Historically, pharmacologic treatment of RA has traditionally followed the pyramid approach. That is, treatment starts with corticosteroids/non-steroidal anti-inflammatory drugs, then progresses to disease-modifying anti-rheumatic drugs and finally to biologic response modifiers, if persons are non-responsive to the previous drugs. Arthritis Rheum 2002, 46, 328-346.

Mixed Connective Tissue Disease.

Mixed connective tissue disease (MCTD) is an uncommon, specifically defined, overlap syndrome characterized by clinical features of systemic lupus erythematosus, scleroderma, and polymyositis/dermatomyositis with very high titers of circulating antinuclear antibody to a ribonucleoprotein antigen. The criteria for MCTD are published as Alarcon-Segovia D, Cardiel M H. J Rheumatol 1989, 16, 328-34.

Systemic Lupus Erythematosus.

Systemic lupus erythematosus (SLE) is a rheumatic and autoimmune disease characterized by fatigue, body aches, skin rash, neurologic complaints, and effects on the kidney, lung, and heart. The American College of Rheumatology (ACR) 1982 Revised Criteria for SLE are published as Tan E M et al., Arthritis Rheum 1982, 25, 1271-7. The ACR 1982 Revised Criteria for SLE Update is published as Hochberg M C. Arthritis Rheum 1997, 40, 1725.

Sjögren's Syndrome. Sjögren's syndrome (SS) is a rheumatic and autoimmune disease characterized by dry eyes and dry mouth, and effects on the joints. The American College of Rheumatology (ACR) Criteria for SS are published as Fox RI et al., Arthritis Rheum 1986, 29, 577-85.

Polymyositis/Dermatomyositis.

Polymyositis/Dermatomyositis (PM/DM) is a rheumatic and autoimmune disease characterized by muscle weakness, difficulty swallowing, rash around eyes, and effects on the heart and lung. The criteria for PM/DM are published as Bohan A, Peter J B. N Engl J Med 1975, 292, 344-7 (first of two parts) and Bohan A, Peter J B. N Engl J Med 1975, 292, 403-7 (second of two parts); or Bohan A et al., Medicine (Baltimore) 1977, 56, 255-86.

### Fibrogenesis as Dysregulated Wound Healing Response to Lung Epithelial Injury

IPF is a progressive deadly disease that is characterized by gradual fibrosis of the supporting framework, the interstitium, of the lungs. Currently, there is no approved pharmacological treatment available in the United States and lung transplantation is usually the only option to prolong survival. Although the exact pathogenesis of IPF is unknown, a dysregulated wound healing response to lung epithelial injury, which leads to progressive interstitial fibrosis, is a hallmark of the disease. Hereby, transforming growth factor-β (TGF-β) appears to play a central role in mediating induction of stromal genes, which sets off fibrogenesis.

The fibrotic scar is generated, in large part, by activated fibroblasts, which are sometimes referred to as myofibroblasts, based on their expression of alpha smooth muscle action (α-SMA). Lung fibroblasts transdifferentiate into myofibroblasts in response to TGF-β, which is released largely by injured type I or type II alveolar epithelial cells and activated macrophages in fibroblastic foci in lungs affected by idiopathic pulmonary fibrosis. The myofibroblasts produce type I and type III collagen, which compose the fibrotic interstitial scar, and other mesenchymal proteins such as secreted protein acidic and rich in cystein (SPARC), plasminogen activator inhibitor 1 (PAI-1) and fibronectin (FN).

The Mammalian Target of Rapamycin (mTOR)

mTOR and Signaling.

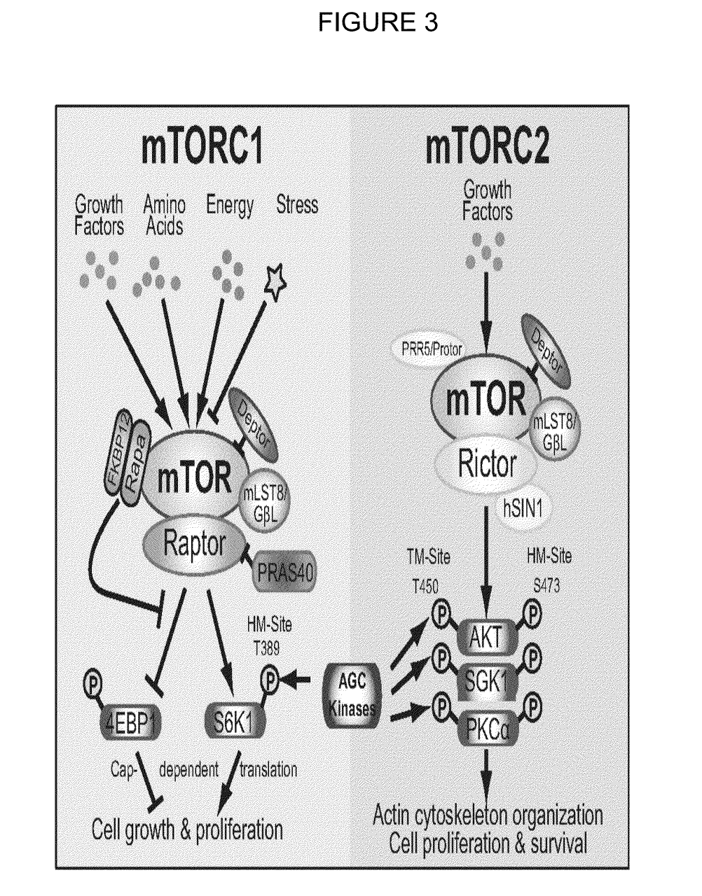

Referring to FIG. 3, the mammalian target of rapamycin (mTOR) is a 289-kDa serine/threonine protein kinase, a member of the PI3K-related kinase (PIKK) family (Sarbassov, D. D., et al., Phosphorylation and regulation of Akt/PKB by the rictor-mTOR complex. Science, 2005. 307(5712): p. 1098-101). The mTOR pathway integrates both extracellular and intracellular signals and acts as a central regulator of cell metabolism, growth, proliferation and survival (Proud, C. G., mTORC1 signalling and mRNA translation. Biochem Soc Trans, 2009. 37(Pt 1): p. 227-31).

To date, two mTOR complexes have been characterized: mTOR complex 1 (mTORC1), a rapamycin-sensitive complex, and mTOR complex 2 (mTORC2), a rapamycin-insensitive complex (Hay, N. and N. Sonenberg, Upstream and downstream of mTOR. Genes Dev, 2004. 18(16): p. 1926-45). In mammalian cells, mTOR resides in two physically and functionally distinct signaling complexes, mTORC1 and mTORC2 (Huang, S. K., et al., Prostaglandin E2 induces fibroblast apoptosis by modulating multiple survival pathways. FASEB J, 2009). mTORC1 consists of at least five components: (i) mTOR, the catalytic subunit of the complex; (ii) Raptor; (iii) mLS8; (iv) PRAS40; and (v) Deptor. mTORC1 phosphorylates the ribosomal S6K1 (protein S6 kinase 1) and 4E-BP1 (eukaryotic translation initiation factor eIF4E binding protein 1) proteins, which regulate growth and protein synthesis, respectively (Jacinto, E., et al., Mammalian TOR complex 2 controls the actin cytoskeleton and is rapamycin insensitive. Nat Cell Biol, 2004. 6(11): p. 1122-8). Rapamycin and rapalogs inhibit phosphorylation of S6K1 and 4E-BP1 through inhibition of mTORC1 (Serini, G., et al., The fibronectin domain ED-A is crucial for myofibroblastic phenotype induction by transforming growth factor-beta1. J Cell Biol, 1998. 142(3): p. 873-81).

mTORC2 is a rapamycin-insensitive complex, consisting of six different known proteins: (i) mTOR; (ii) Rictor; (iii) mSIN1; (iv) Protor-1; (v) mLST8; and (vi) Deptor. Rictor and mSIN1 mutually stabilize each other, thus establishing the structural foundation of the complex (Jacinto, E., et al., Mammalian TOR complex 2 controls the actin cytoskeleton and is rapamycin insensitive. Nat Cell Biol, 2004. 6(11): p. 1122-8). Deptor negatively regulates mTORC2 activity, and mLST8 is also necessary for the stability and activity of the complex. mTORC2 has been shown to mediate the phosphorylation of Akt on Ser 473, leading to activation of the Akt pathway, which initiates several cancer-related cellular responses, including increased cell growth and proliferation, a shift to glycolytic metabolism, and increased cell migration (Wu, L. and R. Derynck, Essential role of TGF-beta signaling in glucose-induced cell hypertrophy. Dev Cell, 2009. 17(1): p. 35-48). There is also evidence suggesting that mTORC2 phosphorylates PKCα and regulates the organization of the actin cytoskeleton (Bhagwat, S. V. and A. P. Crew, Novel inhibitors of mTORC1 and mTORC2. Curr Opin Investig Drugs. 11(6): p. 638-45). PI3K also mediates a PDK1-dependent activation of Akt at Thr 308 in response to growth factors, such as insulin growth factor type 1 (IGF-1) (Sarbassov, D. D., et al., Phosphorylation and regulation of Akt/PKB by the rictor-mTOR complex. Science, 2005. 307(5712): p. 1098-101).

The mRNA sequence of human mTOR is published as NCBI Reference Sequence NM_{—}004958.3. The protein sequence of human mTOR is published as NCBI Reference Sequence NP_{—}004949.1 The mTORC1 and mTORC2 complexes contain shared and distinct partner proteins and control a myriad of cellular processes in response to diverse environmental cues. Recent studies have indirectly implicated mTORC2 as a target of TGF-β. In a study by White et al, for example, the authors showed that TGF-β activates Akt (Ser473) in fetal lung fibroblasts, which led to induction of fibronectin (FN) expression and increased generation of the alternatively spliced extra type III domain A (EDA) fibronectin variant (White, E. S., et al., Control of fibroblast fibronectin expression and alternative splicing via the PI3K/Akt/mTOR pathway. Exp Cell Res, 2010. 316(16): p. 2644-53; Chang, W. T., et al., Triptolide and chemotherapy cooperate in tumor cell apoptosis. A role for the p53 pathway. J Biol Chem, 2001. 276(3): p. 2221-7). The EDA spliced variant of FN is expressed by fibroblasts in fibroblastic foci in IPF lung and regulates TGF-β-mediated conversion of fibroblasts into myofibroblasts (Ashcroft, T., J. M. Simpson, and V. Timbrell, Simple method of estimating severity of pulmonary fibrosis on a numerical scale. J Clin Pathol, 1988. 41(4): p. 467-70). The authors further showed that rapamycin inhibited expression of FN and EDA-FN, without affecting Akt (Ser473) phosphorylation, and thereby implicated mTORC1 as the key regulator of FN and EDA-FN expression by fibroblasts exposed to TGF-β.

Another recent study showed that glucose regulates cell hypertrophy in fibroblasts and epithelial cells through TGF-β-mediated activation of mTOR (mTORC1 and mTORC2) (Wu, L. and R. Derynck, Essential role of TGF-beta signaling in glucose-induced cell hypertrophy. Dev Cell, 2009. 17(1): p. 35-48). The discovery of ATP-competitive mTORC1/2 inhibitors was recently reported by several research groups, but a selective mTORC2 inhibitor has yet to be developed. A few dual active-site mTOR inhibitors have progressed to clinical trials for cancer (Bhagwat, S. V. and A. P. Crew, Novel inhibitors of mTORC1 and mTORC2. Curr Opin Investig Drugs. 11(6): p. 638-45). The PP242, Ku-0063794, and Wye-354 compounds are commercially available (Chemdea, Ridgewood, N.J.). Because Akt is constitutively phosphorylated at Ser473 in IPF fibroblasts and Akt is activated by TGF-β, mTORC2 possibly mediates both constitutive Akt phosphorylation (Ser473) in IPF fibroblasts and its activation by TGF-β. Blocking Akt activation with an active-site mTOR inhibitor might succeed in inhibiting the expression of mesenchymal genes, which are downstream of Akt.

### Rapamycin

The bacterial macrolide-derived rapamycin interacts with the cellular protein FKBP12, and this complex directly binds to the mTOR FKBP12-rapamycin-binding (FRB) domain to allosterically inhibit mTORC1. Although the mechanism by which rapamycin inhibits mTORC 1 remains incompletely defined, rapamycin weakens the interaction between mTOR and raptor (regulatory associated protein of mTOR), an mTORC1 regulatory partner, and reduces mTORC1 intrinsic kinase activity. Chronic high-dose rapamycin inhibits mTORC2 signaling in certain cell types by impeding mTORC2 assembly, however.

mTORC1 and mTORC2 contain shared and unique partners. Each complex contains mTOR, mLST8/GβL, and deptor. mLST8/GPL binds the mTOR kinase domain in both complexes but appears more critical for mTORC2 assembly and signaling. Deptor functions as an inhibitor of both complexes. Other partner proteins distinguish the two complexes. mTORC1 contains exclusively raptor and PRAS40. Raptor functions as a scaffolding protein that links the mTOR kinase with mTORC1 substrates to promote mTORC1 signaling. PRAS40 functions in a regulatory capacity. In contrast, mTORC2 contains exclusively rictor (rapamycin-insensitive companion of mTOR), mSin1, and PRR5/protor. Rictor and mSin1 promote mTORC2 assembly and signaling; the function of PRR5/protor remains obscure.

mTORC1 senses and integrates diverse extra- and intracellular signals to promote anabolic and inhibit catabolic cellular processes. Growth factors and nutrients (e.g. amino acids, energy) promote mTORC1-dependent protein synthesis, cell growth (increase in cell mass/size), cell proliferation, and cell metabolism. Conversely, insufficient levels of these factors, or signals of cell stress, blunt mTORC1 action to maintain cellular biosynthetic rates appropriate for suboptimal cellular conditions. Reduced mTORC1 signaling also promotes macroautophagy, a degradative process that enhances cell survival in the face of decreased nutrient availability via the breakdown of cell constituents into amino acids and other small molecules. TORC1 in yeast and mammals also promotes “ribosome biogenesis,” a process whereby mTORC1 increases the transcription of ribosomal RNAs and proteins to augment cellular protein biosynthetic capacity.

Raptor binds directly to mTOR signaling (TOS) motifs on downstream targets, including S6K1 (ribosomal S6 protein kinase 1) and 4EBP1 (eukaryotic initiation factor (eIF) 4E-binding protein 1) (as well as PRAS40 and Hif1α), thus linking them to the mTOR kinase. The TOS motif is required for mTOR/raptor-mediated phosphorylation of S6K1 on its hydrophobic motif (HM) site (Thr389) and 4EBP1 on multiple sites (Thr37/46, Thr70, Ser65). Raptor mutation within its raptor N-terminal conserved domain abrogates 4EBP 1 binding and mTORC 1-mediated 4EBP1 phosphorylation in vitro while retaining mTOR interaction, thus underscoring the importance of the raptor-4EBP1 interaction for mTORC1 signaling. As the avidity of the raptor-mTOR interaction increases during nutrient and growth factor insufficiency (when mTORC1 signaling is low), raptor may possess opposing cell condition-dependent functions in mTORC1 regulation.

The serine-threonine protein kinase Akt (also known as protein kinase B) represents the first identified substrate of mTORC2. Akt promotes cell proliferation, cell survival, and cell migration and controls various metabolic processes. Full activation of Akt in response to growth factor-mediated PI3K signaling requires dual phosphorylation on its activation loop site (Thr308) by PDK1 and HM site (Ser473) by mTORC2. mTORC2 also phosphorylates the HM sites on SGK1 (Ser422) and protein kinase Cα (PKCα; Ser657). As the mTORC1 substrate S6K1 and the mTORC2 substrates Akt, PKCα, and SGK1 represent AGC kinases, an emerging theme in mTOR signaling is that mTORC1 and mTORC2 phosphorylate members of the AGC kinase family.

Active-Site mTOR Inhibitors

The molecules Torin 1, PP242 (a pyrazolopyrimidine), INK128, Ku-0063794, and WAY-600 are representative members of the class of selective and ATP-competitive inhibitors of mTOR. Thoreen C. C. et al., J. Biol. Chem., 2009, 284, 8023-8032; Feldman M. E. et al., PLoS Biol., 2009, 7, e38; Garcia-Martinez J. M. et al., Biochem. J., 2009, 421, 29-42; Yu K. et al., Cancer Res., 2009, 69, 6232-6240.

Unlike rapamycin, these molecules are dual mTOR inhibitors and, thus, inhibit both mTORC1 and mTORC2. Active-site or dual mTOR inhibitors have been synthesized, that target the ATP binding site of mTOR and which block both mTORC1 and mTORC2, but there are no specific mTORC2 inhibitors.

Unlike PI3K family inhibitors such as LY294002, these molecules inhibit mTOR with selectivity relative to PI3Ks and protein kinases. To distinguish these molecules from the allosteric mTORC1 inhibitor rapamycin, we are calling them active-site mTOR inhibitors for TOR kinase (catalytic) domain inhibitors.

PI3Ks catalyze the synthesis of the phosphatidylinositol (PI) second messengers PI(3)P, PI(3,4)P₂, and PI(3,4,5)P₃ (PIP₃). In the appropriate cellular context, these three lipids control diverse physiological processes including cell growth, survival, differentiation, and chemotaxis. The PI3K family comprises 15 kinases with distinct substrate specificities, expression patterns, and modes of regulation. The class I PI3Ks (p110α, p110β, p110δ, and p110γ) are activated by tyrosine kinases or G protein-coupled receptors to generate PIP₃, which engages downstream effectors such as the Akt/PDK1 pathway, the Tec family kinases, and the Rho family GTPases. The class II and III PI3Ks play a key role in intracellular trafficking through the synthesis of PI(3)P and PI(3,4)P₂. The PI3K-related kinases (PIKKs) are protein kinases that control cell growth (mTORC1) or monitor genomic integrity (ATM, ATR, DNA-PK, and hSmg-1).

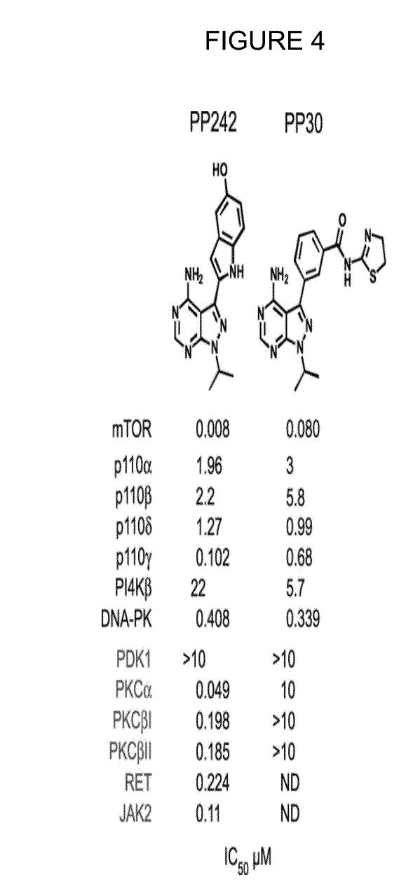

As seen in FIG. 4, PP242 and PP30 inhibit mTOR in vitro with half-maximal inhibitory concentrations (IC₅₀ values) of 8 nM and 80 nM, respectively. As expected for active-site inhibitors, PP242 and PP30 inhibit mTOR in both mTORC1 and mTORC2. Referring to FIG. 4, both compounds are selective within the PI3K family, inhibiting other PI3Ks only at ≧about 10-fold higher concentrations. Apsel B. et al., Nat Chem Biol, 2008, 4, 691-699 tested PP242 against 219 purified protein kinases at a concentration 100-fold higher than its mTOR IC₅₀ value revealing selectivity with respect to the protein kinome; most protein kinases were unaffected by this drug, and only four—PKC-alpha, PKC-beta, RET, and JAK2—were inhibited more than 80%. Feldman M. E. et al., PLoS Biol., 2009, 7, e38 determined IC₅₀ values for PP242 against these kinases in vitro using purified proteins. In these assays, and referring to FIG. 4, PP242 was relatively inactive against PKC-beta, RET, or JAK2 but inhibited PKC-alpha with an in vitro IC₅₀ of 50 nM. These data indicate that PP242 is a selective active-site inhibitor of mTOR.

### Pirfenidone

Pirfenidone, currently approved outside of the United States for the treatment of idiopathic pulmonary fibrosis, is an orally active synthetic antifibrotic agent that inhibits fibroblast, epidermal, platelet-derived, and transforming beta-1 growth factors (according to the National Cancer Institute's dictionary), thereby slowing down progression of the fibrosis, while stabilizing lung function. Pirfenidone also inhibits DNA synthesis and the production of mRNA for collagen types I and III, resulting in a reduction in radiation-induced fibrosis. Pirfenidone does not appear to act on any of the mTOR complexes.

### Utility of the Present Invention

The inventors of the present invention identified pathways existent in IPF fibroblasts and TGF-β-stimulated normal fibroblasts, which mediate expression of stromal genes and found that the mTOR/Akt pathway is activated in IPF fibroblasts and in TGF-β-stimulated normal lung fibroblasts. Furthermore, the mTOR pathway, both mTORC1 and mTORC2, was found to regulate stromal gene expression in both fibroblast populations.

In comparison to rapamycin, which inhibits mTORC1, active-site mTOR inhibitors, which inhibit both mTORC1 and mTORC2, proved to be more effective in suppressing basal expression of pro-fibrotic proteins in IPF fibroblasts and the same group of proteins induced by TGF-β from control lung fibroblasts.

Also, specifically blocking the mTOR pathway by turning off Rictor recapitulated the suppressive results seen with PP242 or Ku-0063794.

Moreover, in the murine bleomycin lung fibrosis model, active-site mTOR inhibitor PP242 significantly inhibited lung fibrosis and expression of stromal genes. These data confirm active-site mTOR inhibitors may be effective in the amelioration, treatment or prevention of idiopathic pulmonary fibrosis and other diffuse parenchymal lung diseases.

By amelioration is meant at least an improvement of the symptoms, where amelioration is used to refer to at least a reduction in the magnitude of a parameter, e.g. a symptom, associated with the pathological condition being treated, such as replacement of non-diseased lung tissue with fibrotic tissue. Treatment or amelioration also includes situations where the pathological condition, or at least symptoms associated with, are completely abrogated, e.g., prevented from occurring, or terminated such as that the subject no longer suffers from the pathological condition, or at least the symptoms that characterize the pathological condition.

As mentioned above, in these applications a therapeutically effective amount of an active-site mTOR inhibitor is administered in a therapeutically effective dosing regimen to a subject who is suffering or at risk from suffering from a diffuse parenchymal lung disease. The active-site mTOR inhibitor inhibits at least mTOR Complex 2 (mTORC2) and may also inhibit mTOR Complex 1 (mTORC1).

By “therapeutically effective amount” is meant an amount of an active-site mTOR inhibitor sufficient to produce a desired result, where the desired result is generally an amelioration, treatment or prevention of a diffuse parenchymal lung disease or at least an amelioration, treatment or prevention of one or more symptoms of the diffuse parenchymal lung disease being treated, particularly the replacement of non-diseased lung tissue with fibrotic tissue. In such methods, a therapeutically effective amount of an active-site mTOR inhibitor is administered to a subject, typically according to a dosing schedule, e.g. daily, weekly, monthly and so forth, that is sufficient to ameliorate, treat or prevent the diffuse parenchymal lung disease or at least symptoms of the diffuse parenchymal lung disease.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### Experimental Procedures

The following methods and materials were used in the examples that are described further below.

Cell Culture and Reagents.

Lung fibroblasts were isolated from subjects undergoing surgical biopsy for the diagnosis of interstitial lung disease or lung transplant for IPF, and non-neoplastic tissue was obtained from subjects undergoing surgical lung cancer resection, as described previously (Chang W. T. et al., J Biol. Chem., 2001, 276, 2221-7). Fibroblasts were cultured in Dulbecco's modified Eagle's medium (DMEM) supplemented with 10% fetal bovine serum (FBS) at 5% CO₂ at 37° C., and were used for studies for up to 5 passages after the initial preparation. For each experiment, cells were plated in culture vessels in DMEM complete medium and cultured until 70-80% in confluence, unless otherwise indicated. Cells were subjected to starvation by washing cells twice with 1× phosphate-buffered saline (PBS), followed by adding DMEM/0.1% serum to each well and incubated for an additional 24 hours before further treatment with recombinant human TGF-β1 (2 ng/ml, Sigma, St. Louis, Mo.), recombinant human Wnt3a (25 ng/ml, R&D Systems, Minneapolis, Minn.), PP242 or Ku-0063794 (Chemdea, Ridgewood, N.J.).

Lung tissue was obtained from subjects undergoing surgical biopsy for the diagnosis of interstitial lung disease or lung transplant for IPF, and non-neoplastic tissue was obtained from subjects undergoing surgical lung cancer resection. The tissue was minced into small pieces with a scalpel and digested with type I collagenase (1 mg/ml; Invitrogen) and hyaluronidase (125 units/ml; Sigma) at 37° C. with agitation for 18 h in Dulbecco's modified Eagle's medium (DMEM; Invitrogen) supplemented with 10% fetal bovine serum. The dissociated tissues were incubated without shaking for 5 min at room temperature, followed by the separation of cell-enriched supernatant into a new tube. The cell fraction was centrifuged at 250×g for 5 min, and the pellet was then resuspended in DMEM with 10% fetal bovine serum. Epithelial cells did not, in general, survive more than one passage and were in large part eliminated through trypsinization. Surviving fibroblasts were cultured in DMEM supplemented with 10% fetal bovine serum at 5% CO₂ at 37° C. Each fibroblast culture was frozen at its earliest available passage and was used for studies for up to five passages. For each experiment, cells were plated in culture vessels in DMEM and cultured until 70-80% confluent, unless indicated otherwise. Cells were subjected to starvation by washing cells twice with 1× phosphate-buffered saline (PBS), followed by the addition of DMEM and 0.1% serum to each well and incubation for an additional 24 h. In the experiments for cell death induced by Glu-plasminogen (American Diagnostica Inc., Stamford, Conn.), phenol red-free DMEM was used.

Western Blot Analysis.

Western blot analysis and band intensity quantitation were performed as described previously (Chang W. T. et al., J Biol. Chem., 2001, 276, 2221-7). Briefly, the protein concentration was measured by Bradford assay (Bio-Rad) according to the manufacturer's instructions. An equal amount of protein was separated by SDS-PAGE and transferred to polyvinylidene difluoride membrane. Immunoblotting was performed using primary antibodies for α-SMA (American Research Products, Inc., Belmont, Mass.); SPARC (Biodesign International, Saco, Me.); cleaved caspase-3 (Asp¹⁷⁵), phospho-Akt (Ser⁴⁷³), phospho-GSK-3β (Ser⁹), and GSK-3β (Cell Signaling Technology, Danvers, Mass.); active β-catenin (8E7; Millipore, Billerica, Mass.); total β-catenin (BD Biosciences); PAI-1 (Santa Cruz Biotechnology, Santa Cruz, Calif.); and α-tubulin (used as a loading control; Sigma) overnight at 4° C., followed by incubation with the appropriate horseradish peroxidase-conjugated secondary antibody (GE Healthcare). The blot was visualized by enhanced chemiluminescence (GE Healthcare) and analyzed using a Kodak Image Station 4000R system (Carestream Health, Rochester, N.Y.).

To detect secreted PAI-1, the culture medium was first cleared by centrifugation, and proteins were precipitated in the presence of ammonium sulfate at 50% saturation overnight at 4° C. with gentle agitation. The excess salts were removed by dialysis against lysis buffer used for total lysate preparation, and protein quantitation was performed by the Bradford assay as described above. To suppress endogenous PI3K activity, 10 μm LY293002 or 1 μm wortmannin (Sigma) was used, and dimethyl sulfoxide (Sigma) was used as a control.

Immunohistochemistry Staining of IPF Lung Tissue Sections.

Lung tissues isolated from subjects undergoing surgical biopsy for the diagnosis of interstitial lung disease or lung transplant for IPF were fixed in 4% paraformaldehyde, dehydrated, paraffin embedded, and sectioned at 5-μm. To block endogenous peroxidase activity, sections were treated with 0.3% hydrogen peroxide in methanol for 20 min, preincubated with 5% goat serum, and treated with anti-α-SMA antibody (1:500), anti-type I collagen antibody (1:500), anti-SPARC antibody (1:500), or anti-phosphor-Akt antibody (1:500) for 1 h at 37° C. Then, the sections were incubated with a biotinylated goat secondary antibody, treated with avidin-biotin complex (Elite ABC kit, Vector Laboratories), and stained with diaminobenzidine tetrahydrochloride and hydrogen peroxide. Images were taken and processed using microscopy (Labophot-2, Nikon) equipped with a microscope digital camera system (Olympus).

Immunofluorescent Staining and Nuclear Localization of β-Catenin.

The immunofluorescent staining and visualization of nuclear β-catenin were described previously (Chang W. T. et al., J Biol. Chem., 2001, 276, 2221-7). Briefly, fibroblasts were grown on two-well chamber slides (Thermo Fisher Scientific, Rochester, N.Y.) in complete culture media until 50-60% confluence. Cells were subjected to starvation (0.1% serum) for 24 hours. After washing, fixing, and blocking, cells were then incubated with anti-active β-catenin (8E7, Millipore's Corporate, Billerica, Mass.) at 1:100 dilution in blocking solution for 16 hours at 4° C. The target proteins were visualized with FITC (fluorescein isothiocyanate)-conjugated secondary antibody (Calbiochem, San Diego, Calif.), and DAPI (4′,6-diamidino-2-phenylindole, Molecular Probes, Eugene, Oreg.) was used for nuclear counterstaining Fluorescence images were taken and processed using microscopy (Labophot-2) equipped with Episcopic-fluorescence attachment (EFD-3, Nikon Instruments Inc., Melville, N.Y.).

Fibroblasts were grown on 2-well chamber slides (Thermo Fisher Scientific) in complete culture medium until 50-60% confluent. Cells were subjected to starvation (0.1% serum) for 48 h. After washing with 1×PBS, cells were treated with 100% methanol at −20° C. for 5 min. Cells were then blocked in 1×PBS and 3% normal goat serum (Sigma) for 30 min at room temperature, followed by incubation with anti-active β-catenin at 1:100 dilution in blocking solution for 16 h at 4° C. The target proteins were visualized with fluorescein isothiocyanate-conjugated secondary antibody (Calbiochem), and 4′,6-diamidino-2-phenylindole (Molecular Probes, Eugene, Oreg.) was used for nuclear counterstaining Fluorescent images were taken and processed using a Labophot-2 microscope equipped with an episcopic fluorescence attachment (EFD-3; Nikon Instruments Inc., Melville, N.Y.). For the quantitation of nuclear localized β-catenin, at least total 100 cells were randomly selected from five fields of each stained sample to obtain the percentage of nuclear localization of β-catenin.

Tcf/LEF-Driven Luciferase Reporter Assay.

Lentiviral-based constructs containing Tcf/LEF transcriptional response element (TOPFlash), or mutant (FOPFlash), were transduced into control lung fibroblasts. A green fluorescent protein (GFP)-coded lentiviral construct was co-transduced into cells as an equalizing reference. At 48 hours post infection, cells were placed into 24-well plates, starved, and treated in a triplicate manner with rapamycin, PP242, or Ku-0063794 plus TGF-β (2 ng/ml) or Wnt3a (25 ng/ml) for 24 hours. Reporter activity was determined in a multi-well plate reader by Luciferase Assay System (Promega). Luciferase activity was normalized for infection efficiency by measuring GFP activity from each well.

Nuclear Isolation, Staining, and Fluorescence-Activated Cell Sorting.

To isolate stable cell nuclei from fibroblasts for the staining of intranuclear β-catenin and for analyzing on a flow cytometer, a protocol disrupting cell membrane by detergent (Triton X-100) and maintaining nuclear membrane integrity by magnesium was adopted from the Flow Cytometry Core Laboratory at the NCI ETI Branch (home.ncifcrf.gov/ccr/flowcore/nuclei.pdf). Briefly, cells were collected by gentle scraping and washed twice with cold PBS. Cells were then resuspended in cold nuclear isolation buffer (320 mm sucrose, 5 mm MgCl₂, 10 mm HEPES, and 1% Triton X-100, pH 7.4) and allowed to incubate on ice for 10 min. Nuclear yield and integrity were confirmed by microscopic examination with trypan blue staining. We routinely observed >98% nuclear isolation efficiency (data not shown). Nuclei were pelleted by centrifugation at 2000×g and washed twice with nuclear wash buffer (320 mm sucrose, 5 mm MgCl₂, and 10 mm HEPES, pH 7.4). Isolated nuclei were then incubated overnight with anti-β-catenin antibody (5 μg/ml) or normal mouse IgG (Santa Cruz Biotechnology), followed by a 1-h incubation with fluorescein isothiocyanate-conjugated anti-mouse IgG (2 μg/ml) in nuclear wash buffer plus 1% bovine serum albumin and 0.1% sodium azide. All steps described above were done at 4° C. After extensive washing, nuclei were resuspended in 250 μl of nuclear wash buffer before flow cytometry analysis. Flow cytometry was performed on an Accuri C6 flow cytometer system (Accuri Cytometers, Inc., Ann Arbor, Mich.) using 488 nm excitation and standard fluorescein isothiocyanate emission optics with 10,000 events from each sample, and analysis was performed using FlowJo software (Tree Star Inc., Ashland, Oreg.).

Analysis of Gene Expression.

RNA was extracted from fibroblasts using TRIzol (Invitrogen), and cDNA converted from 5 μg of total RNA was obtained using a SuperScript first-strand synthesis system for reverse transcription-PCR kit (Invitrogen). To control for genomic DNA contamination, additional RNA samples were processed without reverse transcriptase. The reverse transcription product equivalent to 25 ng of total RNA was then added to a real-time quantitative PCR (qPCR) using a Dynamo SYBR Green qPCR kit (Finnzymes, Espoo, Finland) according to the manufacturer's protocol. The following primers were used: human PAI-1,5′-TGGAACAAGGATGAGATCAG-3′ (sense) and 5′-CCGTTGAAGTAGAGGGCATT-3′ (antisense); human α-SMA, 5′-CTGTTCCAGCCATCCTTCAT-3′ (sense) and 5′-CCGTGATCTCCTTCTGCATT-3′ (antisense); and glyceraldehyde-3-phosphate dehydrogenase, 5′-GACCCCTTCATTGACCTCAAC-3′ (sense) and 5′-CTTCTCCATGGTGGTGAAGA-3′ (antisense). The annealing and amplification temperature was 60° C. Real-time qPCR was performed in strip tubes using a StepOne PCR system (Applied Biosystems, Foster City, Calif.) according to the manufacturer's instructions. The specificity of amplified products was suggested by a melting curve resulting in only one peak.

This was further confirmed by agarose gel electrophoresis of the PCR products visualized under ethidium bromide/UV illumination. Target amplifications were compared with the reference amplifications (glyceraldehyde-3-phosphate dehydrogenase) in the same experiment for each reverse transcription product tested. All reactions were carried out in duplicate, and the threshold cycle (Cₜ) values were determined by automated threshold analysis with the StepOne software. The final results are presented as relative-fold change in target gene expression compared with the reference based on the comparative or ΔΔCₜ method. The efficiency of each primer pair was determined by the qPCR procedure from standard dilutions of cDNA (equivalent to 10 pg to 10 ng of total RNA in the reverse transcription reaction). To assess the effect of an inhibitor of the TGF-β1 receptor/ALK5 in suppressing PAI-1 expression, 10 μm SB431542 (Tocris Bioscience, Ellisville, Mo.) was added 1 h prior to the addition of human recombinant TGF-β1 at 2 ng/ml overnight.

Enzyme-Linked Immunosorbent Assay for Secreted PAI-1.

To determine the concentration of PAI-1 in the culture medium of human lung fibroblasts, cells were plated in a 24-well plate at 5×10⁵ cells/well overnight in DMEM and 10% fetal bovine serum. After serum deprivation for 24 h, the culture medium was collected and spun at 10,000×g for 5 min at 4° C. to remove any cell debris. The PAI-1 concentration of each supernatant was determined using a Quantikine human serpin E1/PAI-1 immunoassay kit (R&D Systems, Minneapolis, Minn.) according to the manufacturer's instructions.

Determination of TGF-β Bioactivity.

The active TGF-β concentration in the cultured fibroblasts was assayed using the co-culture method with mink lung epithelial cells transfected with a truncated PAI-1 promoter fused to a firefly luciferase gene (referred to as MLEC-PAI-1-Lux cells; a kind gift from Dr. George Yang, Stanford University) (Abe M et al. (1994) Anal. Biochem. 216, 276-284). Fibroblasts were seeded in 96-well plates at 2.5×10⁴ cells/well in triplicates, along with MLEC-PAI-1-Lux cells at 1.5×10⁴ cells/well in low serum (1%) medium. The use of 1% serum instead of starvation medium with 0.1% serum was needed to support survival of the MLEC-PAI-1-Lux cells. A standard curve of active TGF-β1 was generated in MLEC-PAI-1-Lux cells with serial dilutions of human recombinant TGF-β1 (0-10 ng/ml; Sigma). After incubation for 24 h, the viability of cells was checked microscopically before washing twice with 1×PBS. Cells were then lysed in 1× passive lysis buffer at 50 μl/well (luciferase assay kit, Promega, Madison, Wis.) and incubated with agitation at room temperature for 20 min. 10 μl of cell lysate was analyzed for luciferase activity according to the manufacturer's instructions. Cells from fibroblast-only wells were used for the cell count. The mean values of luciferase activity from triplicates were then converted into concentrations of TGF-β in picograms/number of cells using a standard curve obtained with human recombinant TGF-β1, normalized with cell numbers. The concentration of total TGF-β1 in the culture medium for corresponding fibroblasts was determined using a Quantikine human TGF-β1 immunoassay kit (R&D Systems) according to the manufacturer's instructions.

Murine Model of Bleomycin-Induced Pulmonary Fibrosis.

Six-week-old male C57BL/6 mice weighing between 20 to 23 g were obtained from The Jackson Laboratory (Bar Harbor, Me.) and all care was in accordance with the National Institutes of Health Guide for Animal Welfare Act. Mice were administrated 0.1 ml of sterile saline (N=5) or bleomycin (Blenoxane, Novaplus, 1.5 U/kg of body weight; VHA Inc., Nippon Kayaku Co., Tokyo, Japan) at day 0 via intratracheal instillation under anesthesia (Krishna, G. et al., Am J. Pathol. 2001, 158, 997-1004). Mice (N=5) received vehicle (15% PVP) or PP242 daily by oral route at 30 mg/kg of body weight starting the day prior to bleomycin treatment for six days a week. All mice were sacrificed on day 14 and the right lungs were first fixed by inflation with buffered 10% formalin solution, then incubated in the same solution for 24 hours. After paraffin-embedding, sections were processed, and stained with hematoxylin and eosin (H&E) for light microscopy. The left lungs were frozen in liquid nitrogen for subsequent analysis of hydroxyproline content.

Hydroxyproline Assay to Measure Collagen Content.

To estimate the total amount of collagen deposited as an indicator of pulmonary fibrosis, the measurement of hydroxyproline content (Woessner, J. F., Jr., Arch Biochem Biophys., 1961, 93, 440-7) of whole lung was conducted for each sample according to the manufacture's instruction (Biocolor Ltd., UK). Briefly, Sirius red reagent was added to each lung homogenate (20 ml) and mixed for 30 min. The collagen-dye complex was precipitated by centrifugation at 16,000 g for 5 min, washed with ethanol, and dissolved in 0.5 M NaOH. The concentration of collagen in each sample was measured as absorbance at 540 nm and values extrapolated from a known standard curve as per manufacturer's instructions.

Histological Scoring of Fibrosis.

The severity of fibrosis from each lung section was assessed by Ashcroft score (Ashcroft T. et al., J Clin Pathol, 1988, 41, 467-70). Briefly, the grade of lung fibrosis was scored on a scale from 0 to 8 by examining 30 randomly chosen fields per sample at a magnification of ×100. Criteria for grading lung fibrosis were as follows: grade 0, normal lung; grade 1, minimal fibrous thickening of alveolar or bronchiolar walls; grade 3, moderate thickening of walls without obvious damage to lung architecture; grade 5, increased fibrosis with definite damage to lung structure and formation of fibrous bands or small fibrous masses; grade 7, severe distortion of structure and large fibrous areas; grade 8, total fibrous obliteration of fields. If there was difficulty in deciding between two odd-numbered categories, the field would be given the intervening even-numbered grade. Scoring of the saline+vehicle and saline+PP242 groups both gave values of zero.

RNA Interference.

We constructed a lentivirus-driven β-catenin small hairpin RNA (shRNA) expression plasmid from the pLKO.1 vector (22), targeting to β-catenin (5′-CGGGATGTTCACAACCGAATT-3′; pLKO.1-shβ-Cat), SPARC (5′-AACAAGACCTTCGACTCTTCC-3′; pLKO.1-shSPARC), or a scrambled sequence (5′-GTTCTCCGAACGTGTCACGTT-3′; pLKO.1-Scr). pLKO.1-shβ-Cat, pLKO.1-shSPARC, or pLKO.1-Scr was transduced into cells, followed by puromycin selection at 2 μg/ml for at least 48 h. The efficiency of shRNA knockdown of endogenous β-catenin or SPARC was assessed by Western blot analysis.

Quantitation of Cell Viability and Caspase-3 Activity Assay.

Cell viability was determined by an alamarBlue assay. Briefly, primary fibroblasts were seeded overnight in 96-well plates in triplicates and then subjected to serum starvation (0.1% serum) for 24 h. Cells were left untreated or were treated with Glu-plasminogen at the indicated concentrations for 48 h. alamarBlue (resazurin from Sigma) was added to each well at 1.25 μg/ml for 2-4 h, and the fluorometric assay was done with excitation wavelength at 560 nm and emission wavelength at 590 nm with a fluorescence plate reader (FLUOstar Omega, BMG Labtech, Durham, N.C.). For each assay, data were collected from triplicates and analyzed and are represented as the percentage of viable cells relative to the untreated sample.

Caspase-3 protease activity in Glu-plasminogen-treated lung fibroblasts was determined by the fluorometric reaction using acetyl-Asp-Glu-Val-Asp-7-amino-4-trifluoromethylcoumarin (DEVD-AFC; R&D Systems) as substrate according to the manufacturer's instructions. Briefly, following the induction of cell death by Glu-plasminogen for 24 h, cells were collected by centrifugation and lysed in lysis buffer on ice for 10 min. 50 μl of cell lysate (from 2×10⁶ cells) was mixed with 50 μl of 2× reaction buffer and 10 mm dithiothreitol and 5 μl of 1 mm DEVD-AFC in a 96-well plate. After incubation at 37° C. for 90 min, the release of free AFC cleaved by active caspase-3 proteases was determined using the fluorescence microplate reader with excitation at 400 nm and emission at 505 nm. The level of caspase-3 enzymatic activity in the cell lysate is directly proportional to the fluorescent signal of cleaved AFC.

Statistical Analysis. Data are expressed as the mean±S.D. Two factors one-way analysis of variance and Student's t test were used for intergroup comparison. A probability level of 0.05 (p<0.05) was considered significant.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### IPF Fibroblasts Constitutively Express Sparc, which Activates B-Catenin, Through AKT-Mediated Inhibition of Glycogen Synthase Kinase 3B (GSK-3B)

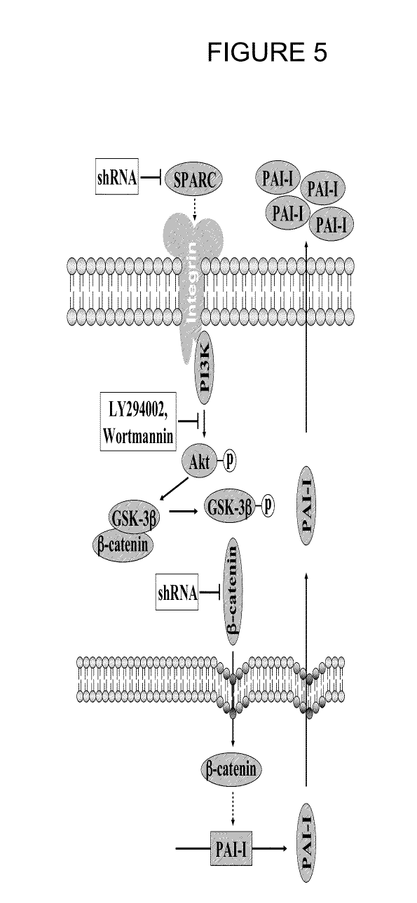

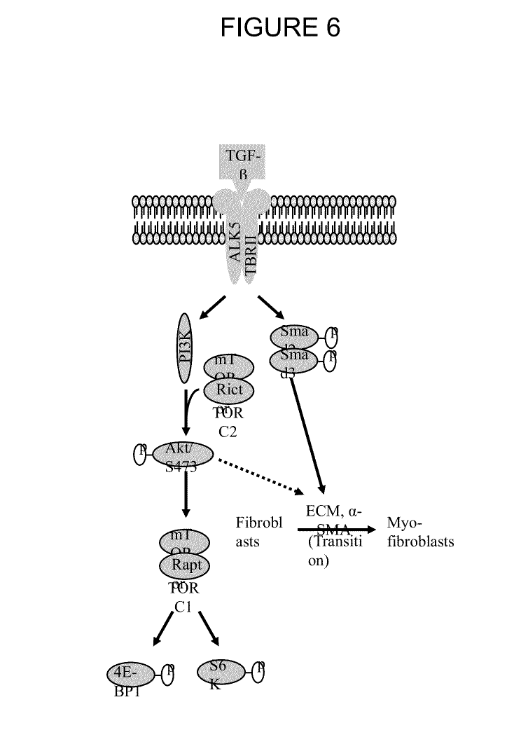

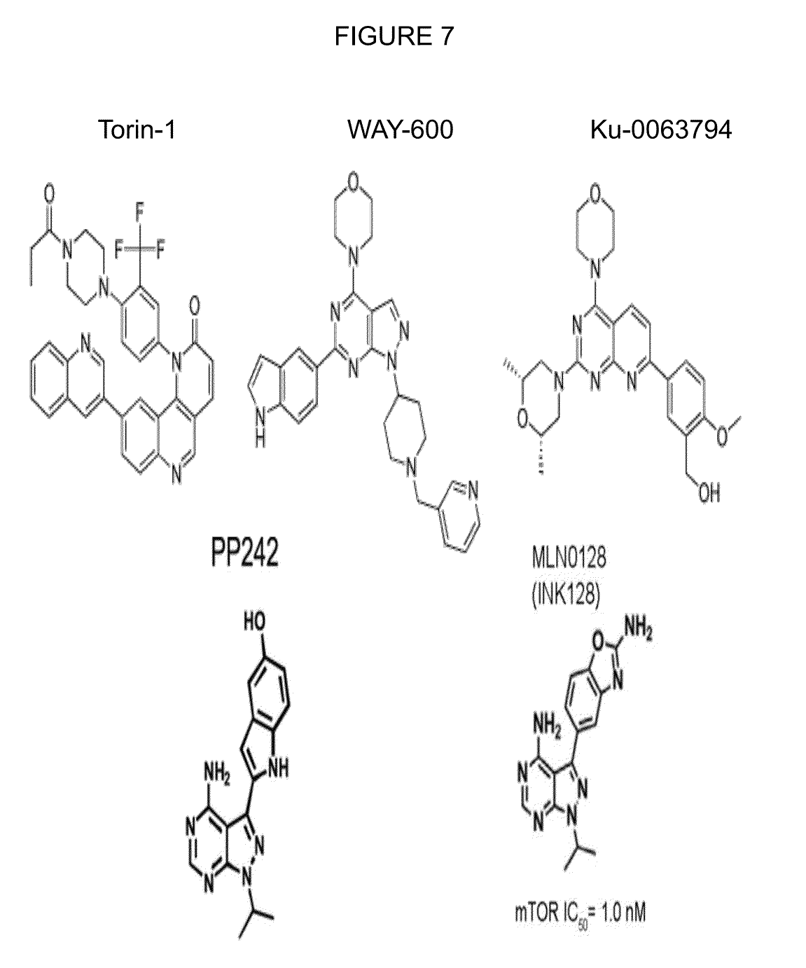

To investigate the phenotype of fibroblasts originating from lungs from subjects suffering from idiopathic pulmonary fibrosis (IPF), fibroblasts were isolated from subjects suffering from IPF subjects and compared with control fibroblasts, which were isolated from tissue taken at the time of lung cancer resection surgery and remote from the cancer. As reported previously, an increase in total α-SMA was observed in IPF fibroblasts versus control fibroblasts. While examining the expression of known matrix regulatory proteins in fibroblasts isolated from control or IPF lung, which have been shown to regulate the wound-healing response, SPARC expression was found to be increased 8-fold in fibroblasts from IPF lung fibroblasts versus control fibroblasts (J Biol. Chem., 2010, 285, 8196-8206). As seen in FIG. 5, enhanced expression of SPARC (secreted protein acidic and rich in cysteine) in IPF fibroblasts stimulated PI3K activity, followed by Akt activation. Akt-mediated phosphorylation of GSK-3 (glycogen synthase kinase-3β) released β-catenin for nuclear translocation and transcriptional activation of PAI-1 (plasminogen activator inhibitor-1). The accumulation of secreted PAI-1 protected IPF fibroblasts from plasminogen-induced apoptosis.

SPARC was more consistently expressed at higher levels in IPF fibroblasts versus control fibroblasts than fibronectin. Type I collagen was consistently expressed at higher levels in IPF fibroblasts, but unlike SPARC, levels varied more with serial passage.

### Example 2

### Dual mTOR Active-Site Inhibitors are More Effective than Rapamycin in Suppressing Constitutive Expression of Mesenchymal Proteins in IPF Fibroblasts and in Response to TGF-β in Normal Lung Fibroblasts

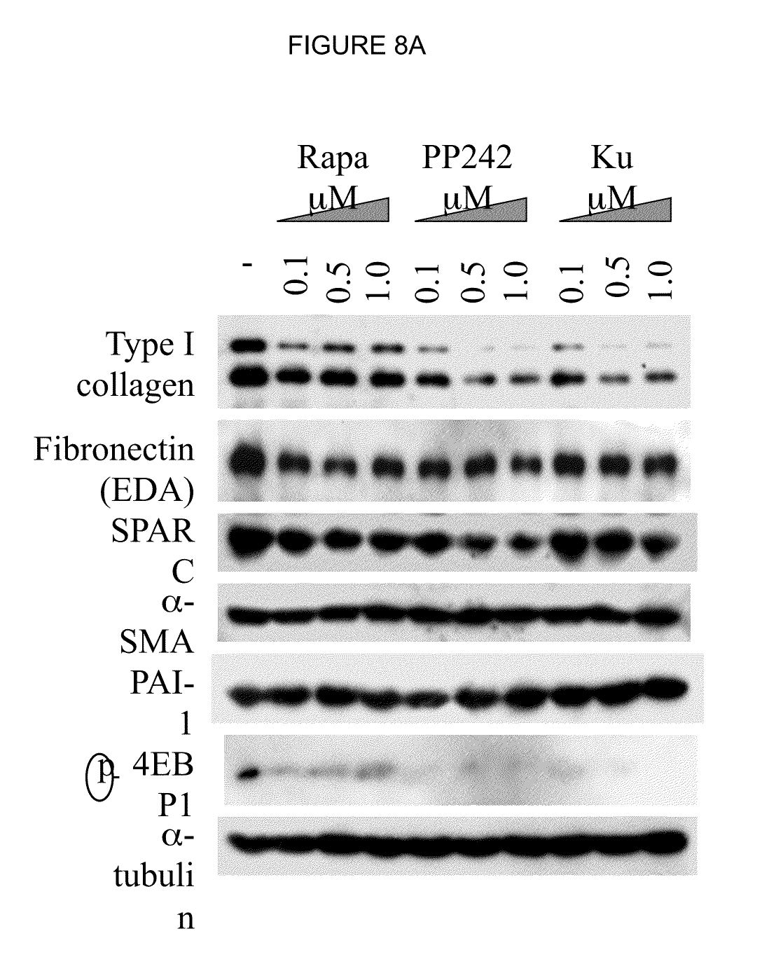

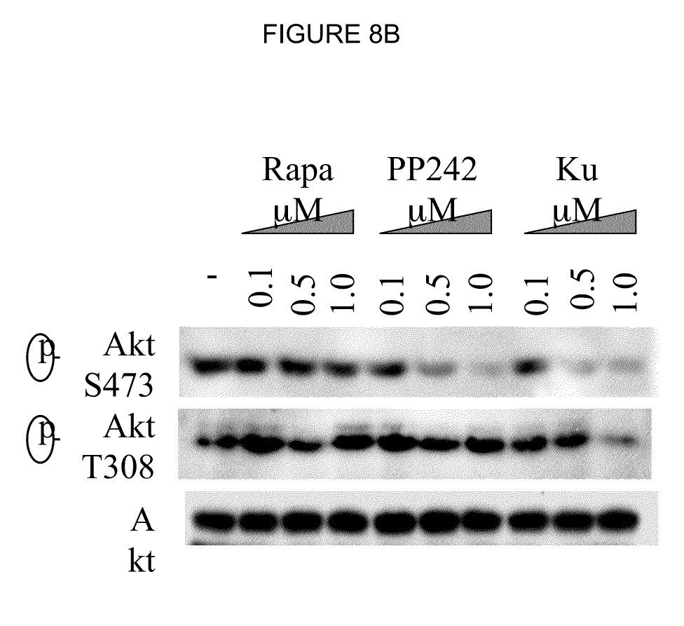

In these studies, we first investigated IPF fibroblasts and found that both PP242 and Ku-0063794, but not rapamycin, suppressed basal Akt (Ser473) phosphorylation in IPF fibroblasts in a dose-dependent manner (FIG. 8A). These doses of rapamycin were 10-100 fold higher than what are used clinically, but mirror the doses that are common for active-site mTOR inhibitors. PP242 and Ku-0063794 were more effective than rapamycin in suppressing basal expression of type I collagen, both inhibiting its expression by over 80% compared to a 50% reduction by rapamycin (FIG. 8A). All inhibitors suppressed FN and SPARC by greater than 50%, but reduced PAI-1 and α-SMA by only 10%-15% of baseline. Cell viability was not compromised by any of these inhibitors at these doses. IPF fibroblasts did not significantly increase stromal protein expression in response to TGF-β, which was likely due to their constitutive high basal expression in IPF fibroblasts (data not shown). All inhibitors blocked the phosphorylation of 4EBP1, which was mTORC1 mediated (FIG. 8A). To establish that Akt (Ser473) phosphorylation was mediated by mTORC2, we showed that PP242 or Ku-0063794, but not rapamycin, inhibited basal Akt (Ser473) phosphorylation in IPF fibroblasts (FIG. 8B). We also looked at their effect on Akt (T308) phosphorylation, which was mediated by PI3K activation of PDK1, and was inhibited by active site mTOR inhibitors (Richard, D. J., J. C. Verheijen, and A. Zask, Recent advances in the development of selective, ATP-competitive inhibitors of mTOR. Curr Opin Drug Discov Devel, 2010. 13(4): p. 428-40). We found that Ku-0063794 was more effective than rapamycin or PP242 in suppressing basal Akt (T308) phosphorylation in IPF fibroblasts (FIG. 8B).

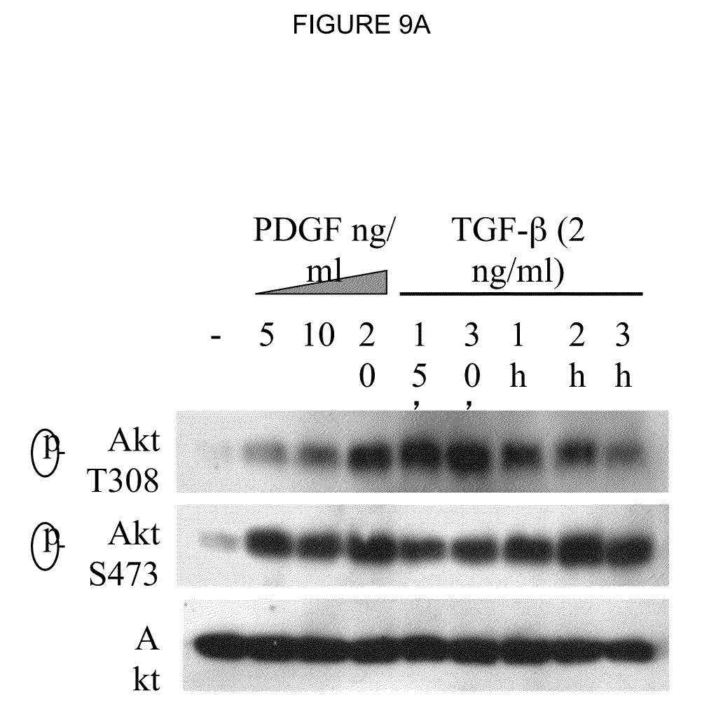

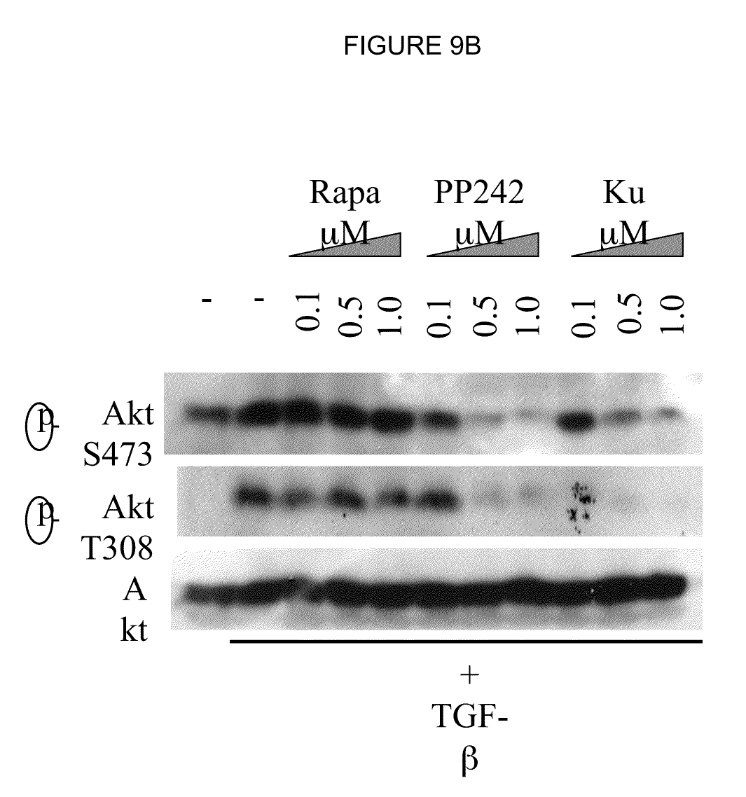

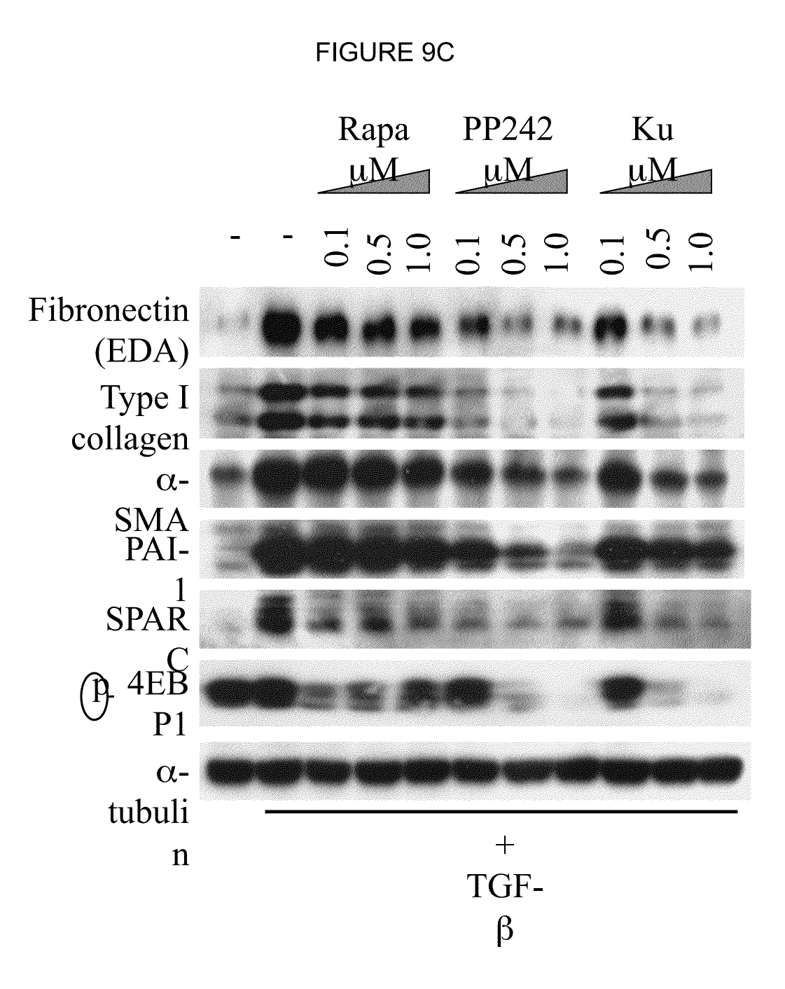

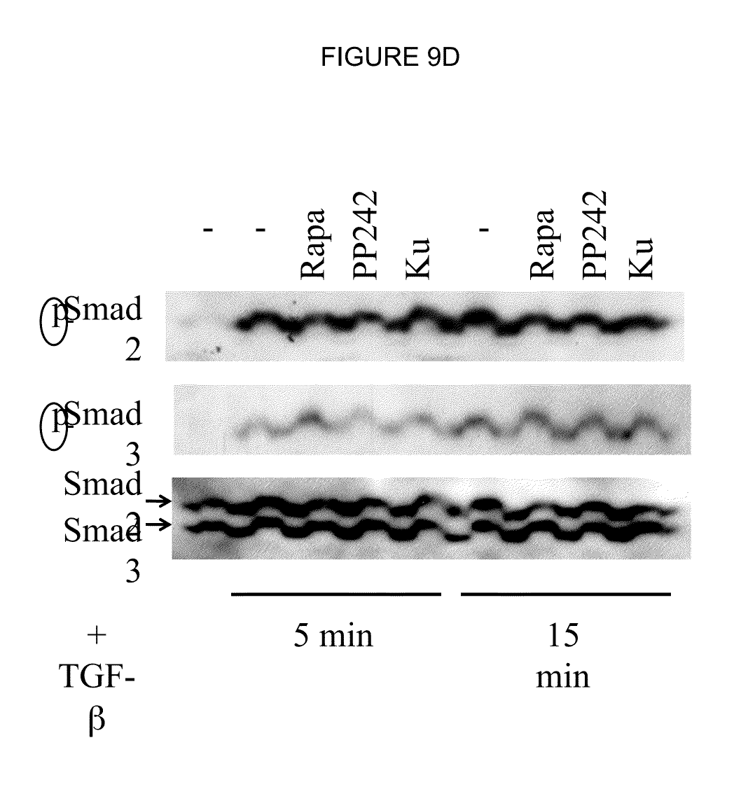

Even though constitutive Akt phosphorylation in IPF fibroblasts does not appear to be mediated by TGF-β alone, there is consensus that TGF-β plays a central role in mediating fibrosis in IPF. In further studies, we examined if inhibition of mTORC1 and/or mTORC2 would limit TGF-β-mediated induction of stromal genes. First, we demonstrated that TGF-β activated Akt in control lung fibroblasts, as we observed activation of both Akt (Ser473) and Akt (T308) at 15′ after TGF-β addition; at 3 h, Akt (Ser473) phosphorylation persisted at similar levels, while Akt (T308) phosphorylation began to decrease (FIG. 9A). PP242 or Ku-0063794, but not rapamycin blocked activation of Akt (Ser473 and T308) (FIG. 9B). We then examined the effect of these inhibitors on TGF-β-mediated induction of stromal genes; as with IPF fibroblasts, we found that PP242 or Ku-0063794 were more effective than rapamycin in blocking expression of type I collagen (FIG. 9C). In fact, PP242 or Ku-0063794 inhibited type I collagen by over 95% in the presence of TGF-β while rapamycin reduced its expression by 45% (FIG. 9C). Also, PP242 or Ku-0063794 blocked induction of FN, SPARC, and α-SMA by 75%-85% while rapamycin inhibited their expression by 30%-50%. Moreover, PP242 inhibited PAI-1 induction by >80% but it was inhibited by 30% in the presence of rapamycin or Ku-0063794 (FIG. 9C). These effects were not mediated by Smad3 or Smad3, as none of the inhibitors blocked TGF-β mediated activation of Smad2 or Smad3 (FIG. 9D).

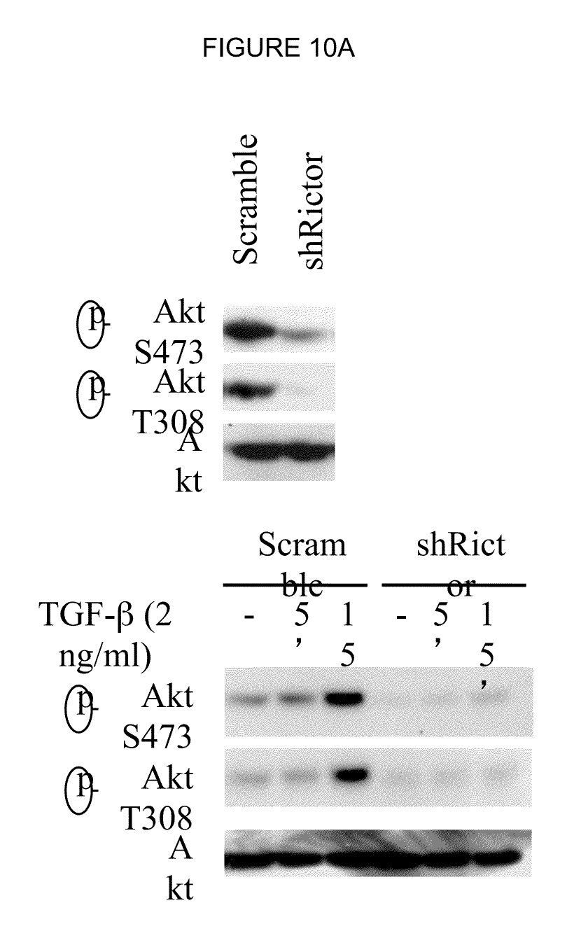

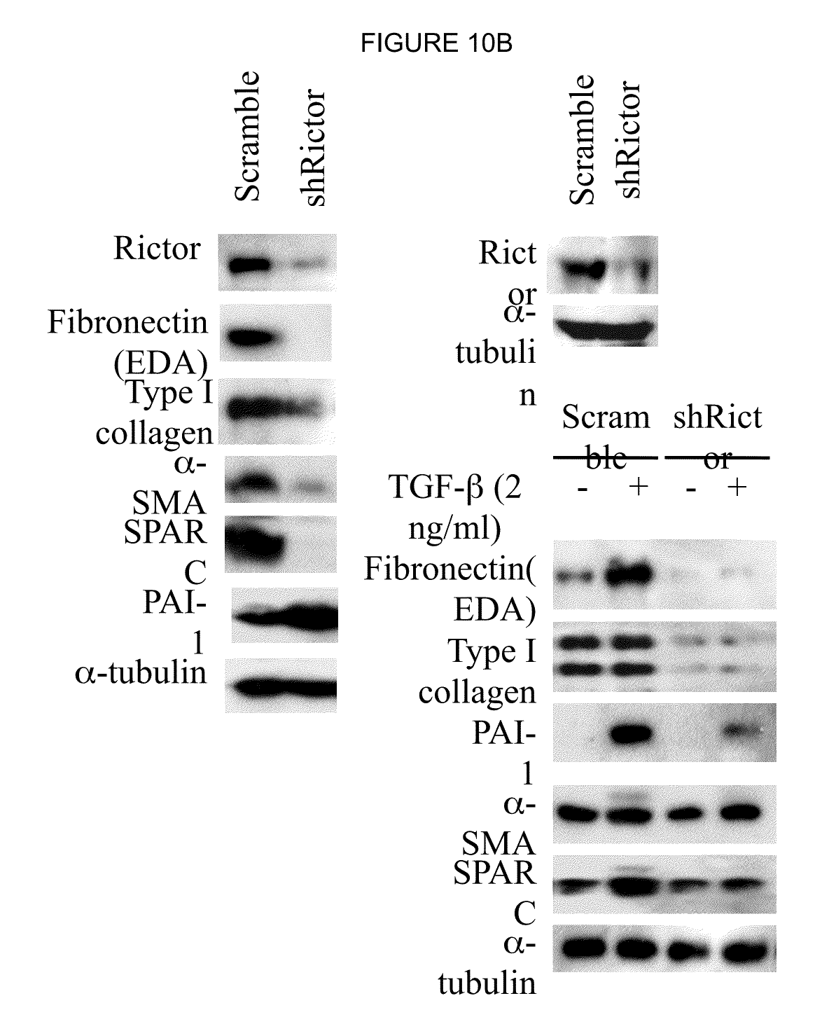

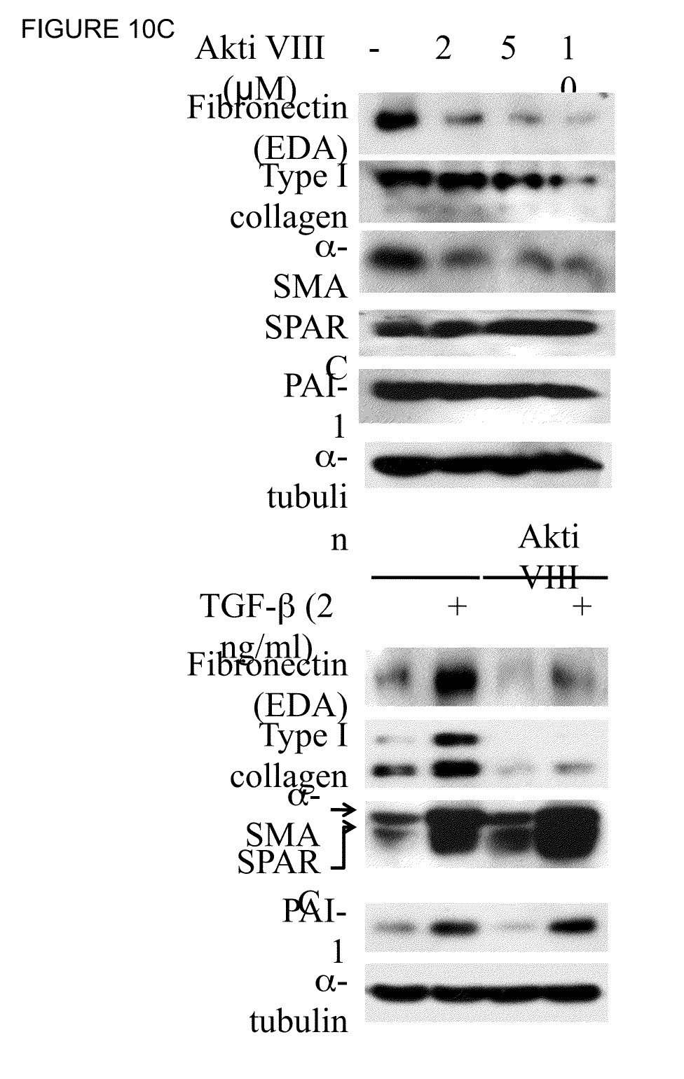

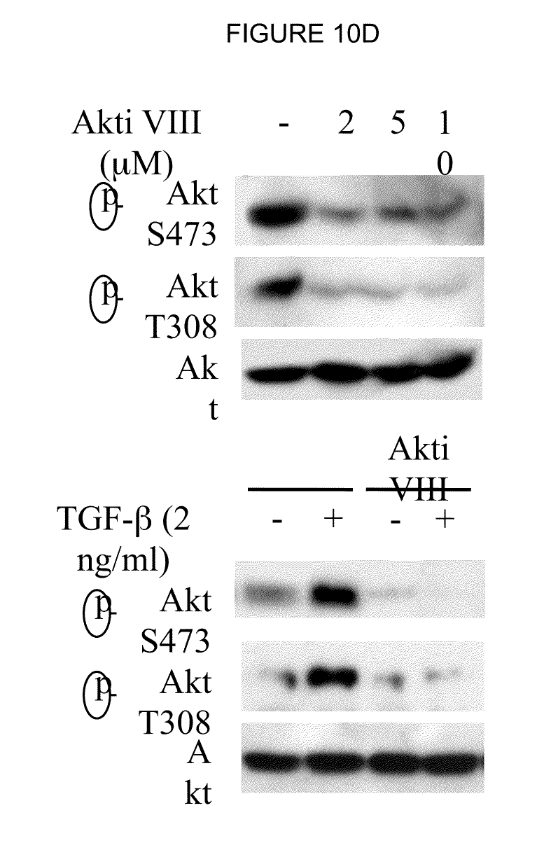

In order to establish that inhibition of mTORC2 mediated, at least in part, suppression of stromal genes constitutively expressed in IPF fibroblasts or induced by TGF-β in control fibroblasts, we examined the effect of downregulation of Rictor by RNA interference, a component of the mTORC2 complex. The downregulation of Rictor suppressed basal Akt phosphorylation (Ser 473 and T308) in IPF fibroblasts and activation by TGF-β in control fibroblasts (FIG. 10A). In IPF fibroblasts, shRictor inhibited FN by 99%, type I collagen by 70%, α-SMA by 95%, and SPARC by 96%; expression of PAI-1, however, was not affected (FIG. 10B). A similar profile was observed in TGF-β-stimulated control fibroblasts except that induction of PAI-1 was inhibited by 80%, but α-SMA was not affected (FIG. 10B). Since Akt is a target of mTORC2, we investigated if inhibition of Akt yielded similar results. We used a specific chemical Akt inhibitor (Akti VIII) and found that it suppressed basal expression of FN, type I collagen, and α-SMA, but not SPARC or PAI-1, in IPF fibroblasts (FIG. 10C); in TGF-β-treated control fibroblasts, Akti VIII inhibited the induction of FN and type I collagen but not α-SMA, SPARC or PAI-1. These results suggest, therefore, that FN and type I collagen are regulated by mTORC2-dependent activation of Akt in IPF fibroblasts but that regulation of SPARC is mediated by Rictor but not Akt; the regulation of PAI-1 or α-SMA is more complex.

In this studies, we show, for the first time, that dual mTOR active-site inhibitors are more effective than rapamycin in suppressing constitutive expression of mesenchymal proteins in IPF fibroblasts and in response to TGF-β in lung normal lung fibroblasts. These data suggest a role for mTORC2 as a mediator of lung fibrosis and suggest that dual mTOR inhibitors, in preference to rapamycin or its analogues, may hold promise for the treatment of fibrotic diseases.

### Example 3

### PP242 Inhibited Bleomycin-Induced Fibrosis when Given Either Simultaneous with Bleomycin or Remote from Bleomycin

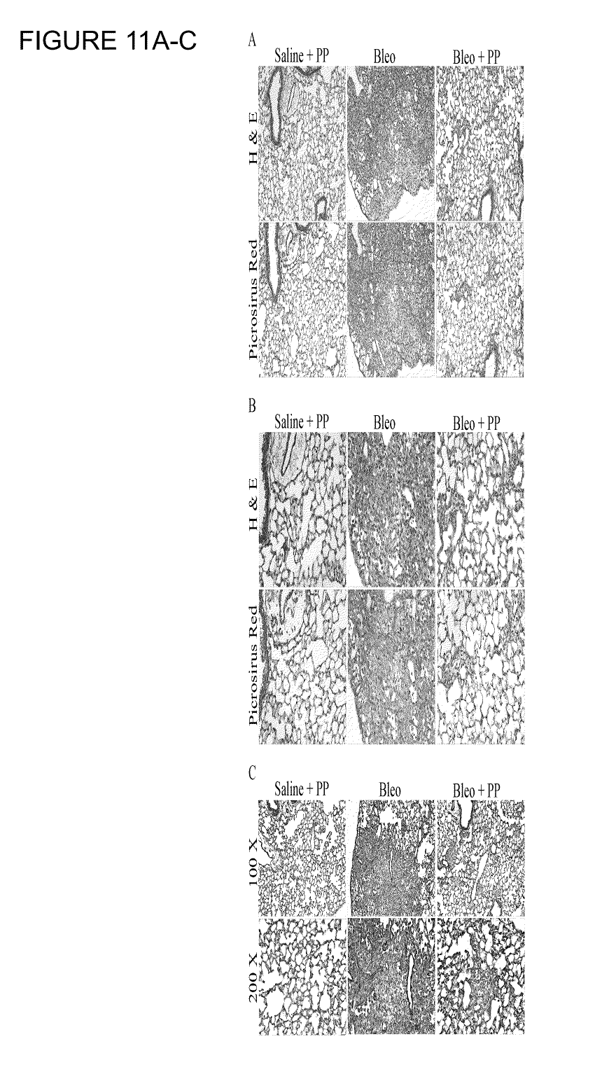

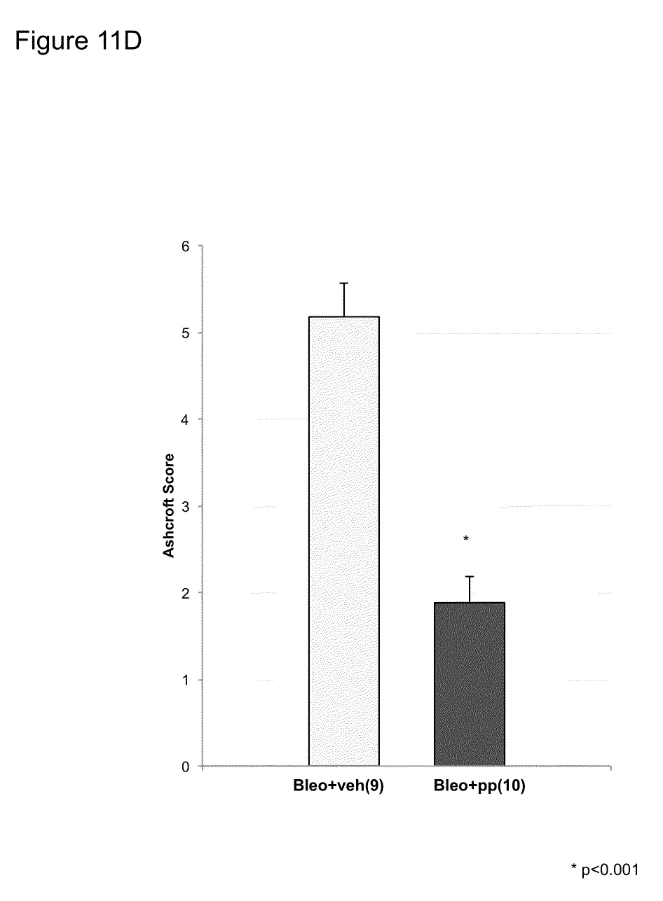

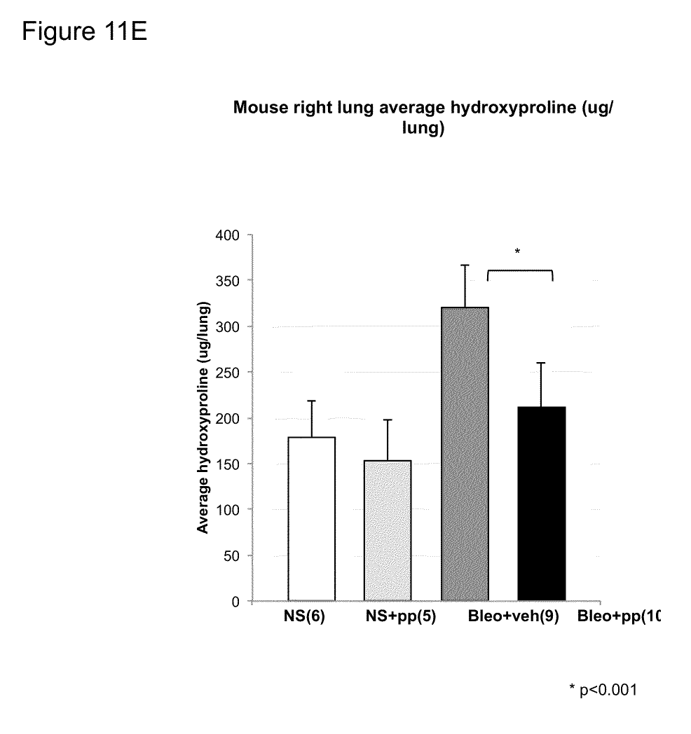

To explore the anti-fibrotic activity of a dual mTOR inhibitor in the murine bleomycin model, we administered PP242 (30 mg/kg) orally, dosages ranging from 30-60 mg/kg, as they have been used in murine cancer models (Shokat). PP242 was given at Day-1 by oral gavage prior to intratracheal instillation of bleomycin (1.5 U/kg), as described previously (Krishna, G., et al., PG490-88, a derivative of triptolide, blocks bleomycin-induced lung fibrosis. Am J Pathol, 2001. 158(3): p. 997-1004) and then daily until sacrifice at Day 14. At the time of harvest, one lung was reserved for histopathologic analysis, i.e., H & E, and Ashcroft score (as assessed by Dr. Gerald Berry, Professor of Pathology, Stanford University); the other lung was used for an analysis of hydroxyproline content. We observed inhibition of bleomycin-induced fibrosis by PP242, as evidenced by a marked reduction in positive staining for picosirius red, a marker of new collagen synthesis (FIG. 11A, B; also, PP242 inhibited accumulation of α-SMA-positive fibroblasts following bleomycin (FIG. 11C). Also, PP242 plus bleomycin compared to bleomycin alone caused a 70% and 40% reduction in the Ashcroft score and hydroxyproline content respectively (FIGS. 11D and 11E). We then examined if genes, which have been shown to mediate fibrosis in the bleomycin model and/or IPF are affected by treatment with PP242; we found that PP242 significantly inhibited bleomycin-mediated induction of SPARC, matrix metalloproteinase 7/matrilysin 1 (MMP-7) and MTS1/S100A4; there was a trend, but not a statistically significant inhibition of PAI-1. We did not observe significant induction of type I collagen, FN, or α-SMA at Day 7 or Day 14 after bleomycin (unpublished results). PP242 was well tolerated; the average weight of animals with PP242 alone was similar to saline control; weight of PP24/bleomycin group was 30-40% more than bleomycin alone (unpublished results).

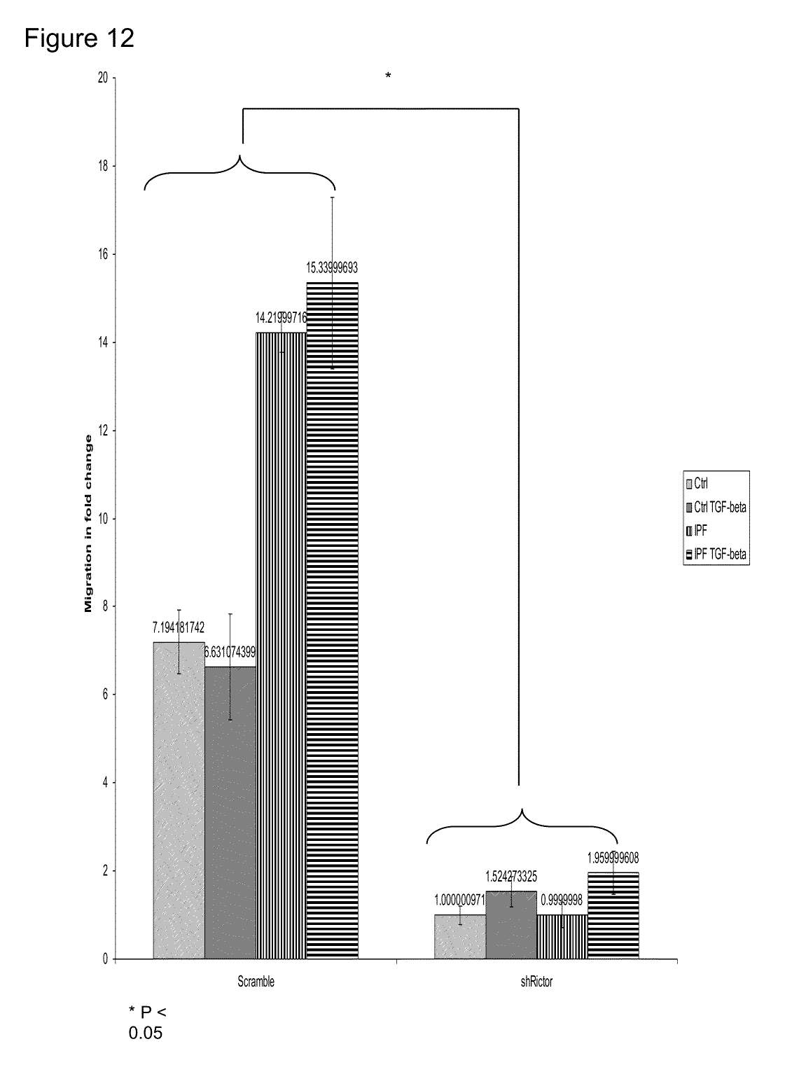

Since the migration of fibroblasts to injured areas is necessary for scar formation in IPF, we investigated whether PP242 or shRictor affected the migration of IPF fibroblasts or control fibroblasts in the absence of presence of TGF-β. We found that PP242 or shRictor significantly reduced the migration of IPF fibroblasts, unstimulated control fibroblasts, or TGF-β-treated fibroblasts; TGF-β did not significantly stimulate migration over baseline, likely because the cells migrated even in serum-depleted conditions (FIG. 12).

### Example 4

### mTORC2 Plays a Part in Regulating Expression of TGF-B-Dependent Stromal-Regulatory Genes

Akt (Ser473) is constitutively active in IPF fibroblasts; furthermore, Akt hyperphosphorylation is mediated by SPARC, which is downstream of TGF-β. Since Akt is a downstream effector of mTORC2, but not mTORC1 in the mTOR complex, inhibition of mTORC2 could be more effective than targeting mTORC1 alone in dampening expression of stromal-regulatory genes in stromal fibroblasts in IPF.

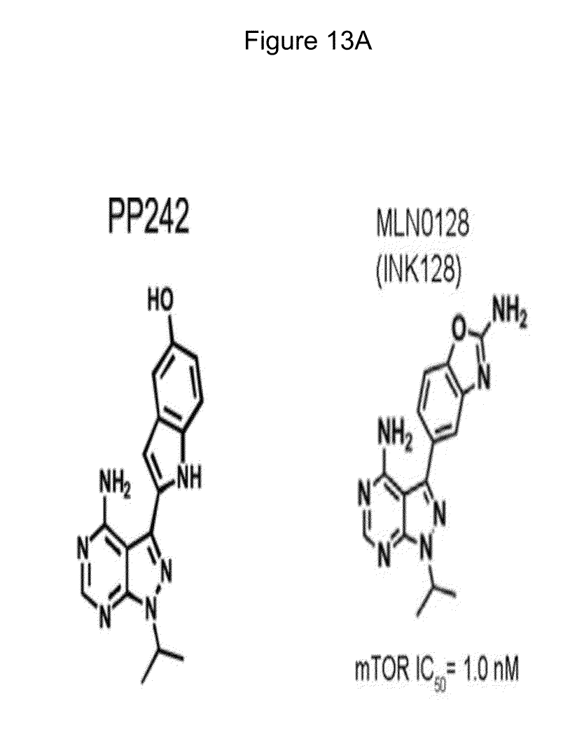

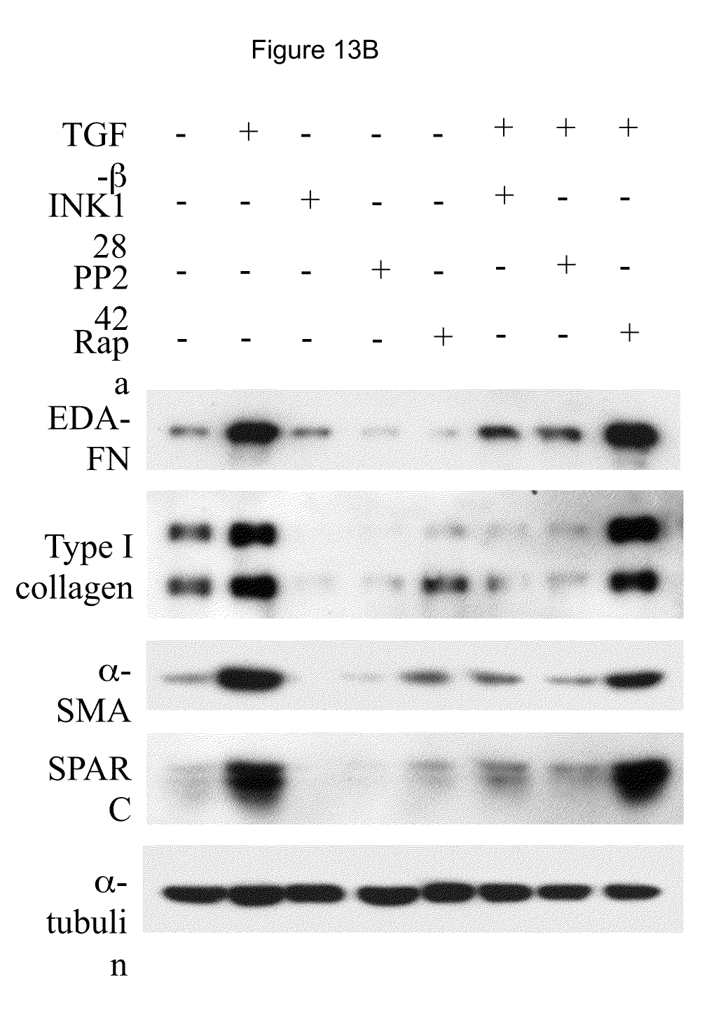

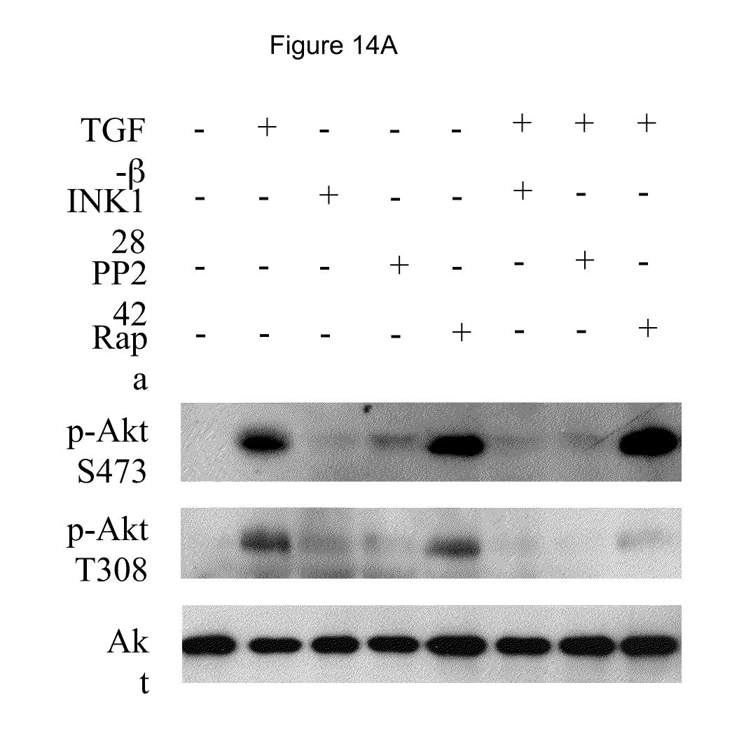

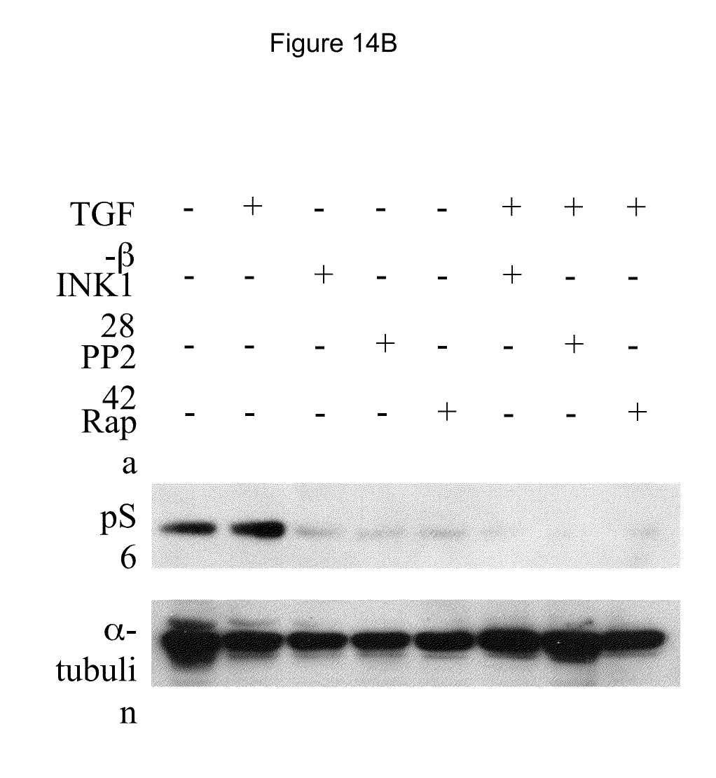

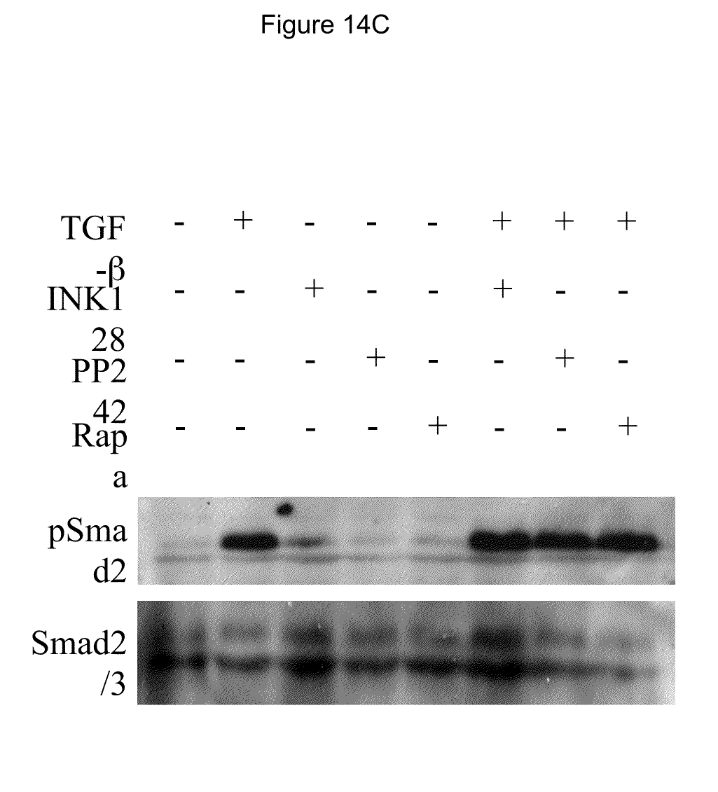

The initial studies were performed with PP-242, a dual mTOR inhibitor, but then advanced to INK128, which is structurally similar to PP242 but is approximately 10-fold more potent (FIG. 13A). In idiopathic pulmonary fibrosis (IPF) fibroblasts, PP242 (2.5 μM) and INK128 (0.2 μM), but not rapamycin (0.1 μM), suppressed the basal and TGF-β-inducible expression of several stromal regulatory proteins by 50%-80%, which include type I collagen, EDA-FN, α-SMA, and SPARC (FIG. 13B). The cultured IPF fibroblasts used in these studies were from different donors because they were used at an early passage, and it was considered clinically relevant to examine samples from different donors; therefore, there are variable levels of expression of basal stromal proteins, which is largely based on the percentage of activated fibroblasts in the population. The dose of each inhibitor selected, i.e., rapamycin, PP242, or INK128, mirrored the effective concentration observed in cellular and mouse studies and was in the range of doses being tested in clinical trials. Also the IC50 of INK128 for suppression of stromal proteins by TGF-β is 30 nM-100 nM. PP242 and INK128 blocked the TGF-β-induced phosphorylation of Akt at serine 473 (FIG. 14A), whereas rapamycin caused hyperphosphorylation of Akt (Ser473). Also, PP242, INK128, and rapamycin inhibited phosphorylation of Akt at threonine 308, which was mediated by PI3K-dependent activation of phosphoinositide-dependent protein kinase 1 (PDK1). Also, as expected, PP242, INK128, and rapamycin blocked phosphorylation of S6 kinase, an mTORC1-dependent target (FIG. 14B). Since the canonical TGF-β pathway involves activation of Smad proteins, next studies investigated whether any of these inhibitors blocked TGF-β-dependent phosphorylation of Smad2 in IPF fibroblasts. Neither PP242, INK128 nor rapamycin blocked activation of Smad2 (FIG. 14C). Cell viability was not compromised by any of these inhibitors.

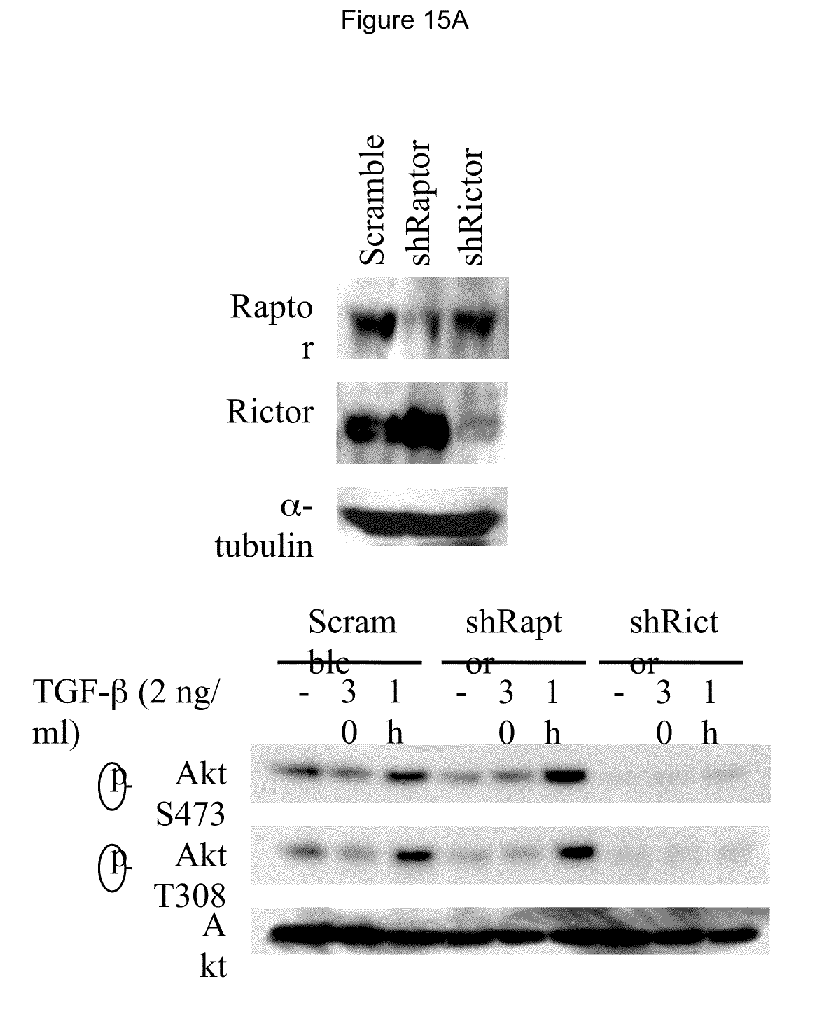

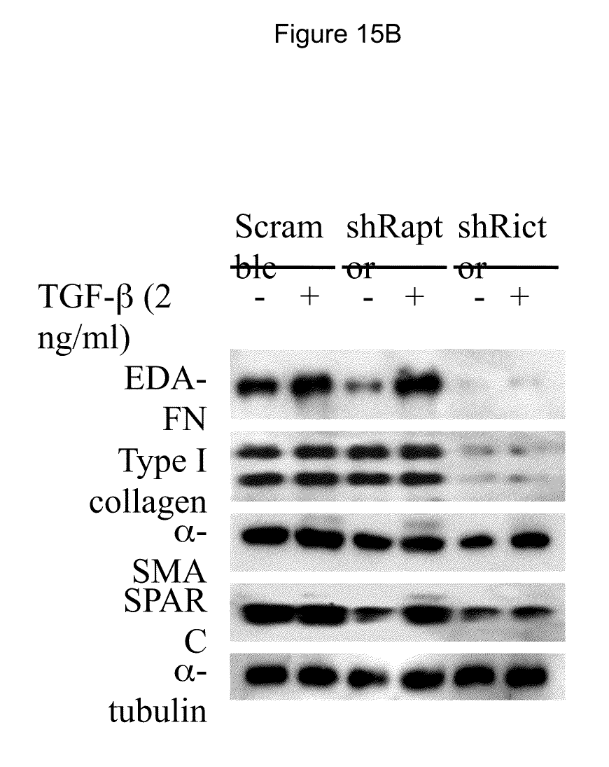

In order to establish that mTORC2 specifically played a part in regulating expression of TGF-β-dependent stromal-regulatory genes, the effect of downregulation of Rictor, a specific component of the mTORC2 complex, or Raptor, an mTORC1-specific component, was investigated. The downregulation of Rictor, but not Raptor, by RNA interference suppressed basal and TGF-β-inducible Akt phosphorylation (Ser 473 and T308) in IPF fibroblasts (FIG. 15A). Also, downregulation of Rictor but not Raptor, inhibited basal and TGF-βinducible FN and type I collagen expression (FIG. 15B). Both shRaptor and shRictor suppressed basal SPARC expression, but only shRictor blocked its induction by TGF-β; (FIG. 15B). The effects on expression of α-SMA were more modest, with an 30% and 40% reduction in basal or TGF-β-induced α-SMA respectively and an 15% and 20% reduction by shRaptor (FIG. 15B). The lesser effect on α-SMA may have been a function of their being a higher percentage of activated fibroblasts in this donor culture.

### Example 5

### Anti-Fibrotic Activity of INK128 in an In-Vivo Murine Lung Bleomycin Model: Prevention and Treatment Strategies

To explore the anti-fibrotic activity of INK128 in vivo, its effect was examined in the murine lung bleomycin model. INK128 (0.75 mg/kg/d IP) was administered (i) as part of a prevention strategy, i.e., INK128 was given on Day −1 prior to bleomycin administration, or (ii) as part of a treatment strategy, i.e., INK128 was started on Day 7 after bleomycin administration. Although oral INK128 is being used in clinical trials with cancer patients, iNK128 was intraperitoneally administered in these studies, because the mice did not tolerate oral gavage with the vehicle (15% polyvinylpyrrolidone K30 (PVP)) that was used to dissolve INK128. A dose of INK128 of 0.75 mg/kg/d was selected based on its efficacy and lack of toxicity in animal murine cancer models. INK128 was given either at Day −1 (prevention) or Day 7 (treatment), with intratracheal bleomycin (1.0 U/kg) at Day 0, using a bleomycin protocol that we have described previously (Krishna G et al., PG490-88, a derivative of triptolide, blocks bleomycin-induced lung fibrosis. Am J Pathol 2001 158:997-1004.)

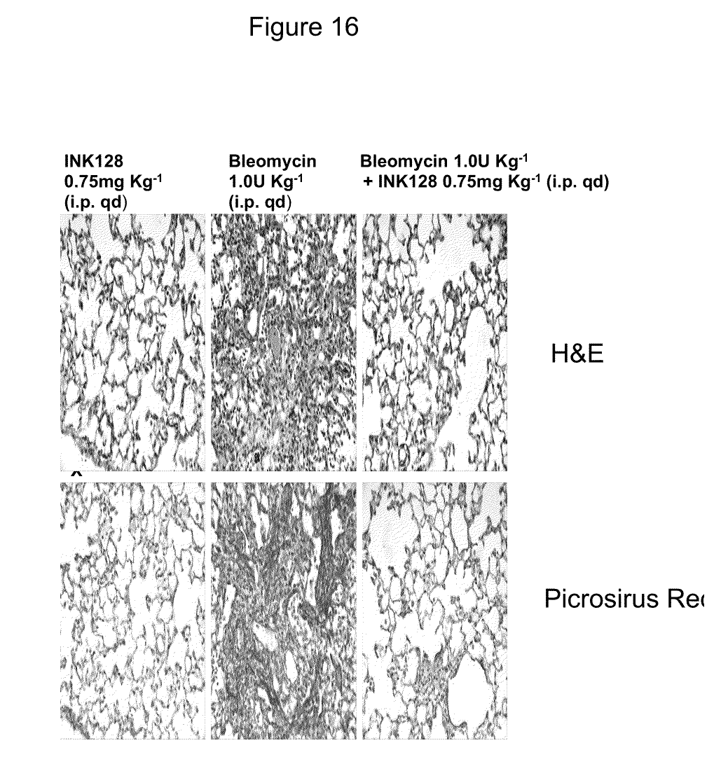

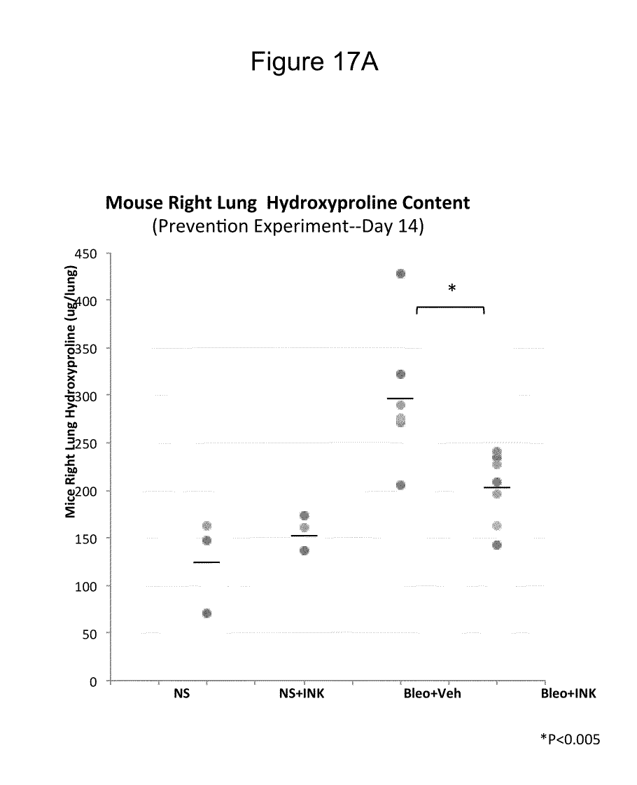

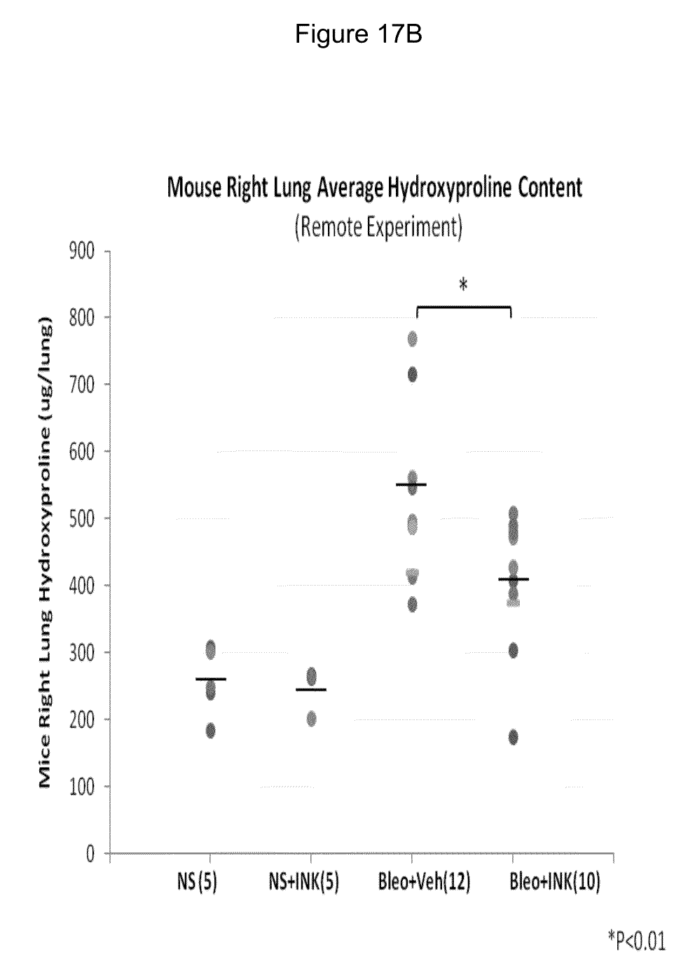

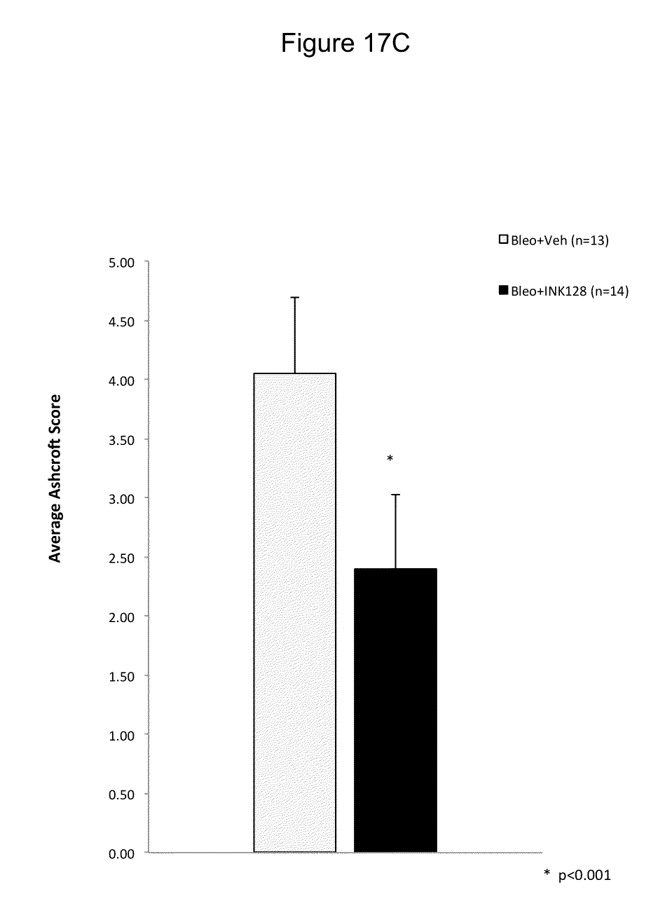

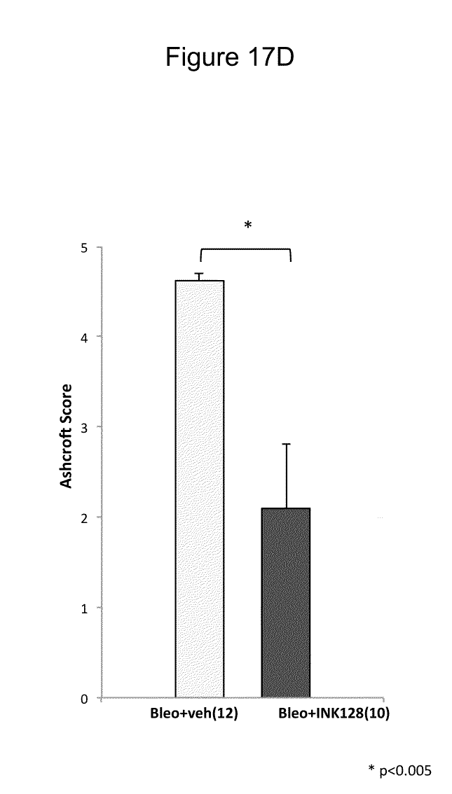

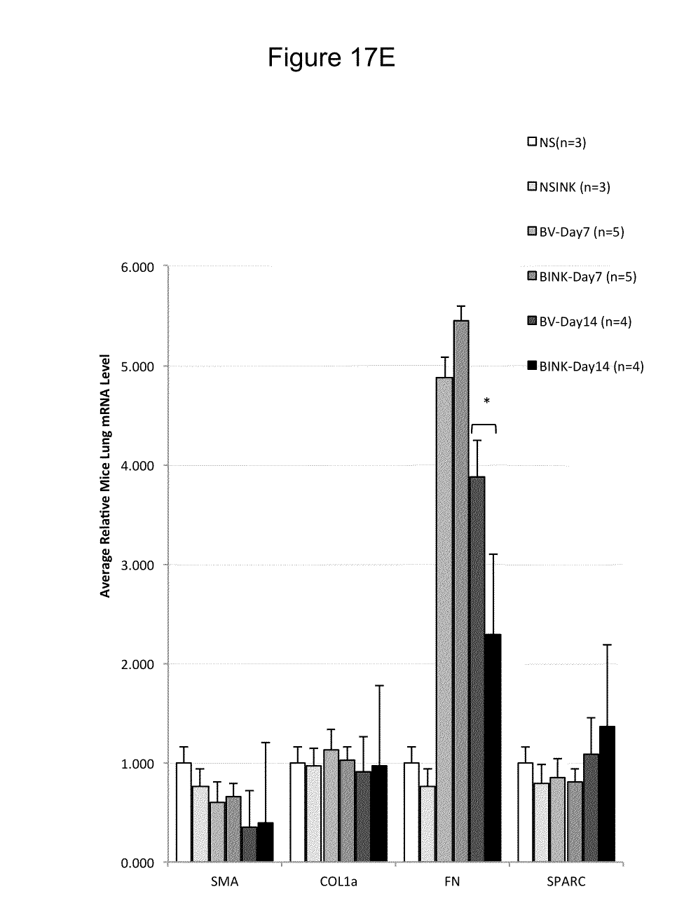

Mice were treated daily with INK128 for 6/7 days, and then sacrificed at Day 14 in the prevention model or at Day 21 in the treatment model. At the time of harvest, one lung was reserved for histopathologic analysis, i.e., H & E, and Ashcroft score (as assessed by Dr. Gerald Berry, Professor of Pathology, Stanford University); the other lung was used for an analysis of hydroxyproline content or gene expression. INK128 was found in both the prevention and treatment model to significantly inhibit inflammation and fibrosis, based on histology, hydroxyproline content, picosirius red staining, and the Ashcroft score (FIG. 16 and FIG. 17).

Next was examined whether INK128 inhibited expression of matrix genes involved in the fibrotic response and those that it had blocked in-vitro in cultured IPF fibroblasts. No significant induction of type I collagen, SPARC, or α-SMA at Day 7 or Day 14 after bleomycin was observed, but this may have been due to slow turnover of these genes, regulation at a post-transcriptional level, and the patchy nature of the injury following bleomycin. However, a significant increase in EDA-FN gene expression was observed at Day 7 and Day 14, which was significantly inhibited by INK128 at Day 14 (FIG. 17E).

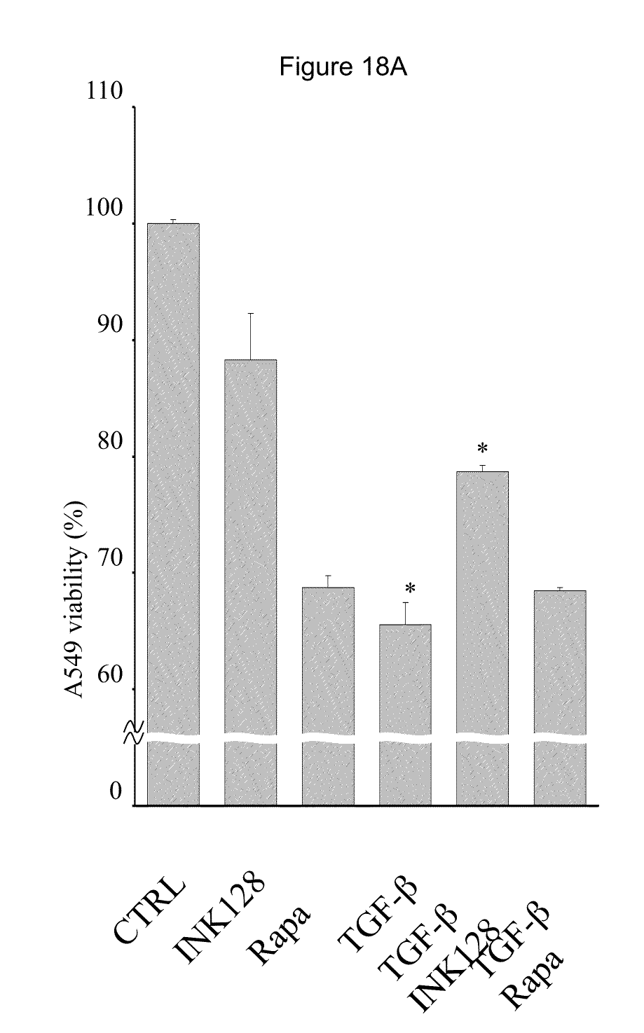

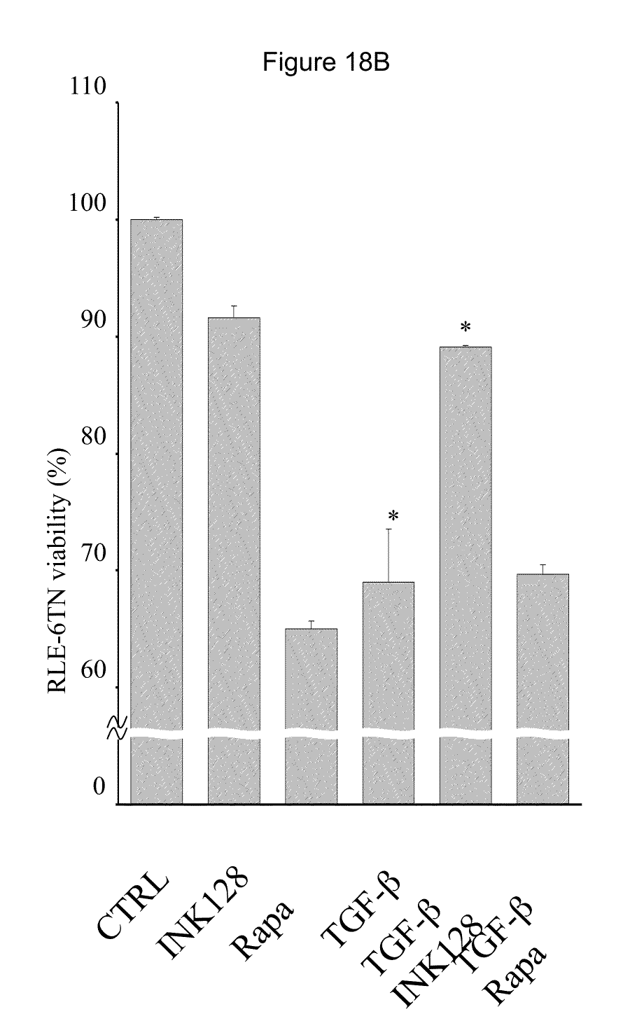

Because matrix proteins and matricellular SPARC are expressed in IPF lung in fibroblastic foci, which are areas of type II alveolar epithelial cell injury subtended by infiltrating TGF-β-activated fibroblasts, epithelial-mesenchymal crosstalk in cultured lung cells was examined. A transwell co-culture model system was utilized, in which TGF-β-treated IPF fibroblasts were co-cultured in a transwell with A549 or RLE-60 TN distal lung epithelial cells. Following 24 h treatment of IPF fibroblasts with TGF-β, the media was changed and the A549 or RLE-6TN cells were placed in a transwell with the fibroblasts in close apposition. After 48 h an Alamar Blue assay was performed to assess cell viability. A 25%-30% reduction in epithelial viability was observed, i.e., A549 or RLE-6TN, following TGF-β treatment of IPF fibroblasts (FIGS. 18A and 18B). Also, rapamycin treatment of IPF fibroblasts reduced epithelial viability in both cell lines to a similar extent and did not protect against TGF-β-induced reduction of epithelial viability (FIGS. 18A and 18B). However, INK128 treatment of IPF fibroblasts did not alone affect A549 or RLE-6TN and it protected against TGF-β inhibition of epithelial viability (FIGS. 18A and 18B). To examine whether SPARC was involved in TGF-β-mediated inhibition of epithelial viability, SPARC was downregulated in TGF-β-treated IPF fibroblasts by RNA interference, using a lentiviral shSPARC construct, J Biol Chem 2010; 285(11):8196-206. SPARC suppresses apoptosis of idiopathic pulmonary fibrosis fibroblasts through constitutive activation of beta-catenin. Chang W, Wei K, Jacobs S S, Upadhyay D, Weill D, Rosen G D

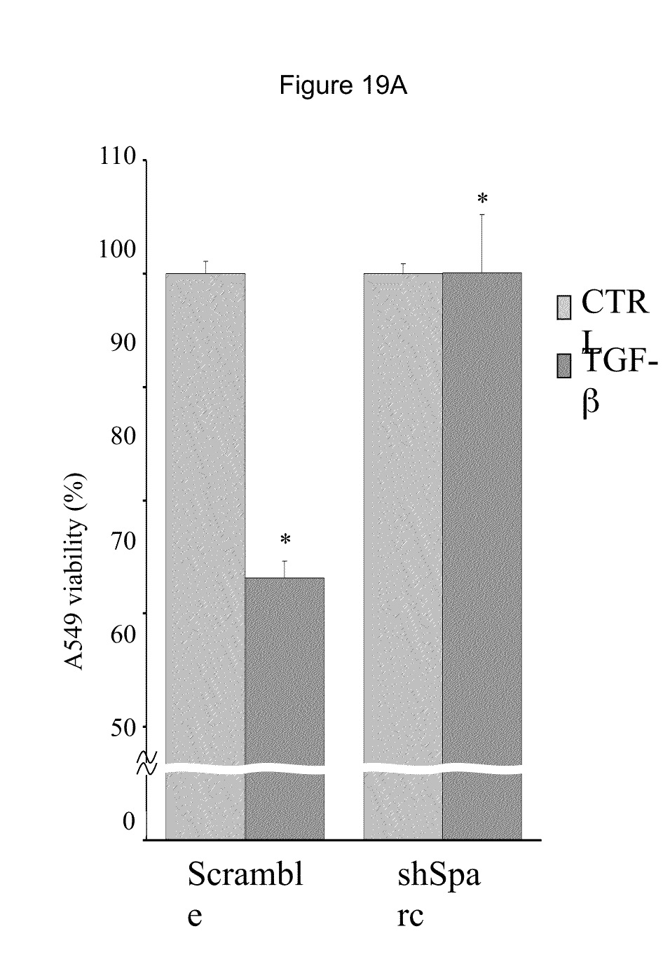

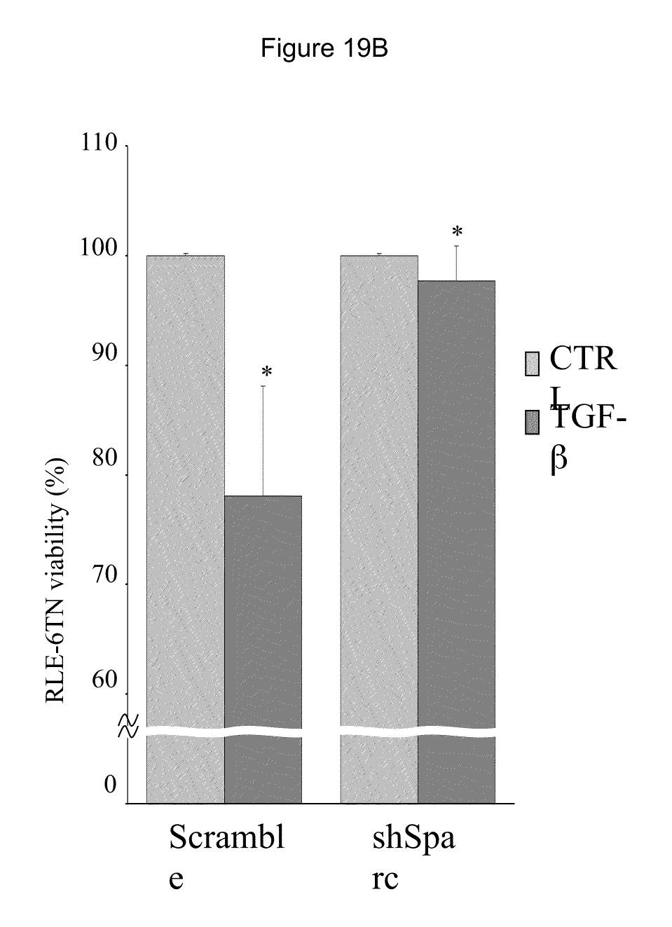

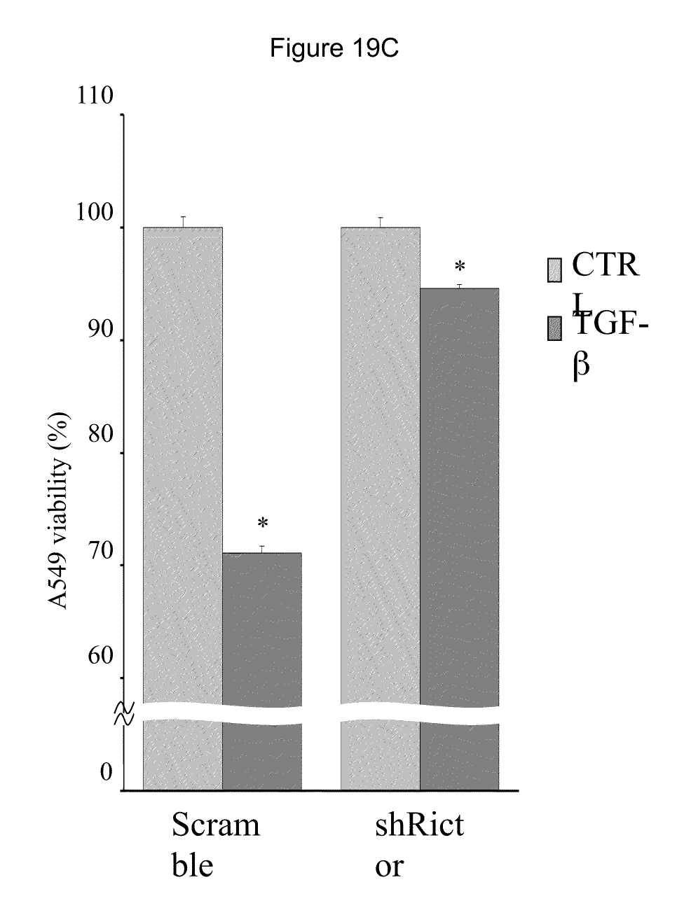

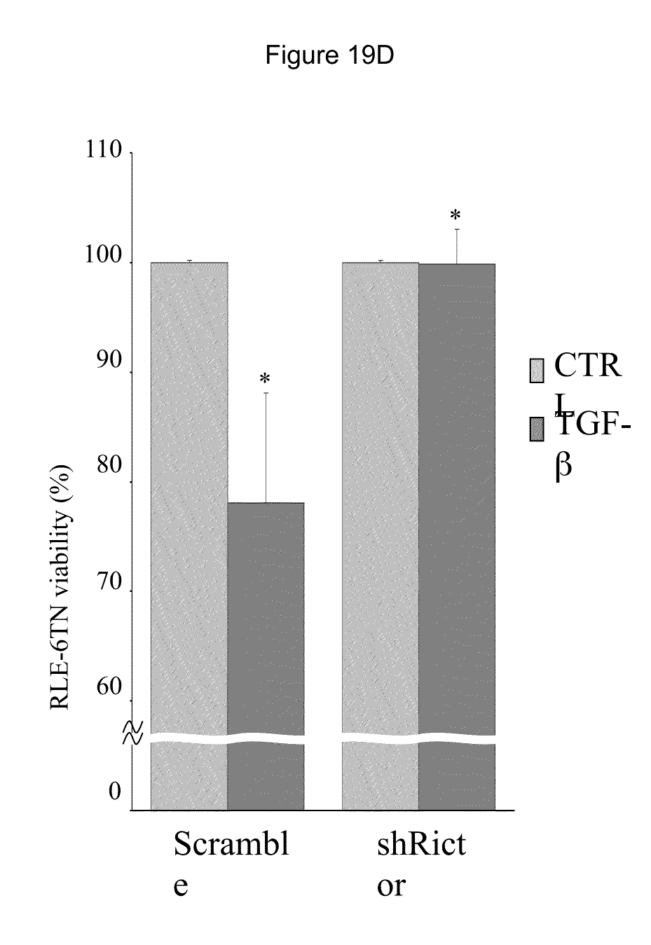

Downregulation of SPARC almost completely restored A549 or RLE-6TN viability following TGF-β treatment of IPF fibroblasts (FIGS. 19A and 19B). Also, since Akt activation by TGF-β was mediated by mTORC2 (see FIG. 15A), it was investigated whether Rictor downregulation in IPF fibroblasts would also restore epithelial viability. Infact, Rictor downregulation by RNA interference also almost completely restored lung epithelial cell viability following TGF-β treatment of IPF fibroblasts (FIGS. 19C and 19D).

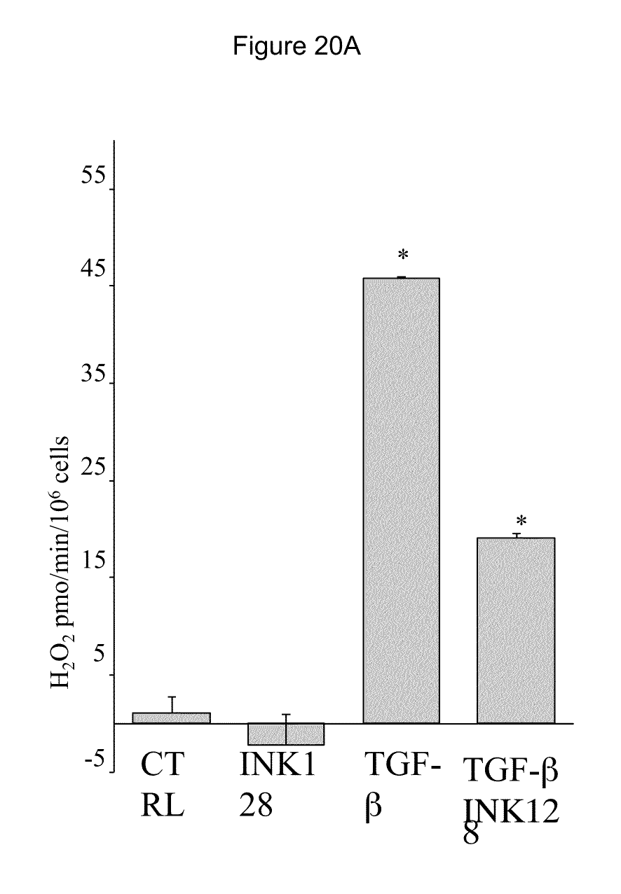

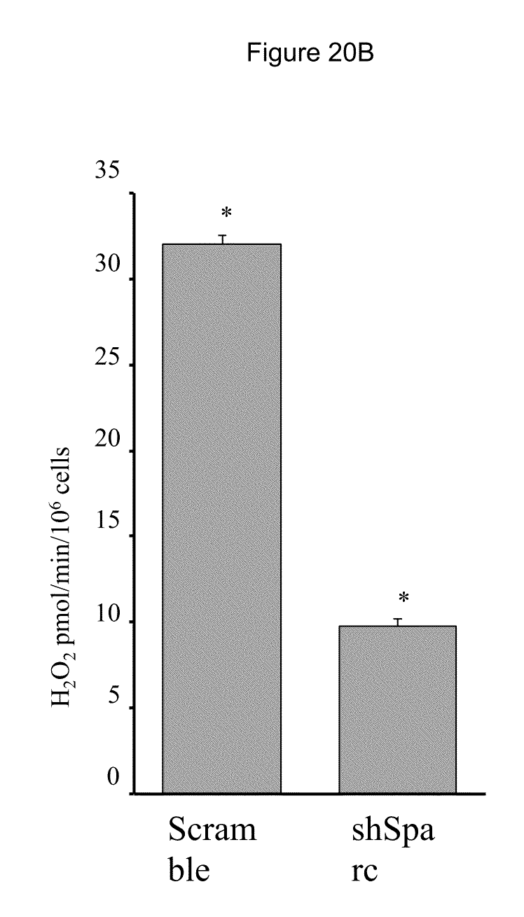

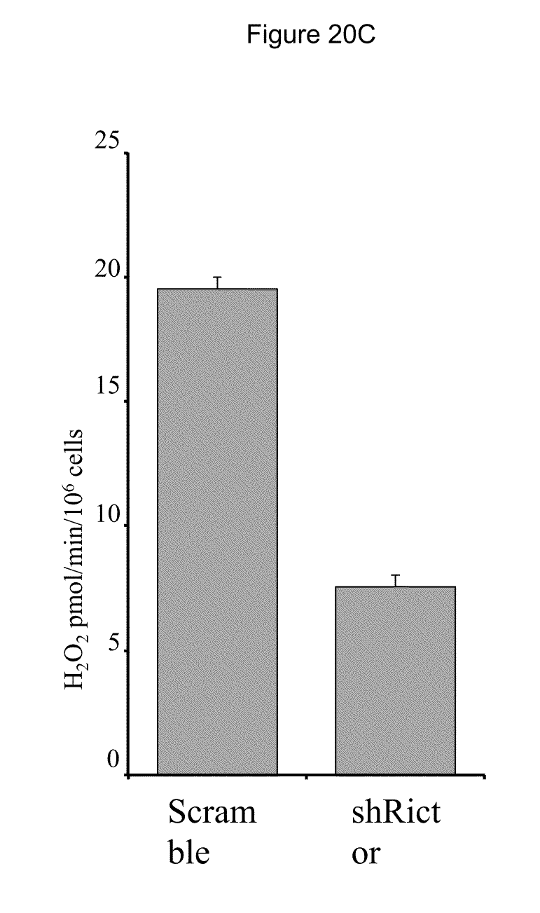

Furthermore, H2O2 release in the presence of INK128 was measured and a 70% reduction in H2O2 release by TGF-β-treated IPF fibroblasts was observed in the presence of INK128 (FIG. 20A). Also, downregulation of SPARC or Rictor suppressed H2O2 production by 68% and 60% respectively (FIGS. 20B and 20C). To confirm that mTORC2 but not mTORC1 mediated H2O2 release from IPF fibroblasts, Raptor was downregulated in IPF fibroblasts, but cell viability was markedly reduced by shRaptor (data not shown).

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It

What is claimed is:

1. A method for ameliorating, treating or preventing diffuse parenchymal lung disease in a subject, comprising administering a pharmaceutical composition comprising a selective active-site inhibitor of mammalian target of rapamycin (mTOR inhibitor) to said subject in a therapeutically effective amount and in a therapeutically effective dosing regimen, wherein the administering is effective to achieve amelioration, treatment or prevention of the diffuse parenchymal lung disease.

2. The method in accordance to claim 1, wherein said diffuse parenchymal lung disease is caused by environmental factors.

3. The method in accordance to claim 1, wherein said diffuse parenchymal lung disease is a collagen vascular disease.

4. The method in accordance to claim 1, wherein said diffuse parenchymal lung disease is an idiopathic interstitial pneumonia.

5. The method in accordance to claim 1, wherein said diffuse parenchymal lung disease is sarcoidosis.

6. The method in accordance to claim 3, wherein said collagen vascular disease is scleroderma.

7. The method in accordance to claim 3, wherein said collagen vascular disease is rheumatoid arthritis.

8. The method in accordance to claim 4, wherein said idiopathic interstitial pneumonia is idiopathic pulmonary fibrosis.

9. The method in accordance to claim 4, wherein said idiopathic interstitial pneumonia is nonspecific interstitial pneumonia.

10. The method in accordance to claim 1, wherein said selective active-site mTOR inhibitor is a pyrazolopyrimidine.

11. The method in accordance to claim 1, wherein said selective active-site mTOR inhibitor is one of Torin 1, PP242, INK128, Ku-0063794 or WAY-600.

12. A method for determining therapeutic efficacy of treatment with an active-site mTOR inhibitor in an subject suffering from a diffuse parenchymal lung disease:
a. Administering a pharmaceutical composition comprising a selective active-site mTOR inhibitor to said subject in a therapeutically effective amount and in a therapeutically effective dosing regimen, wherein the administering is effective to achieve amelioration, treatment or prevention of the diffuse parenchymal lung disease in said subject; and
b. assessing lung function by measuring volume and flow of air that can be inhaled and exhaled by said subject prior and after said administering, wherein an increase in one or both of volume and flow of air that can be inhaled and exhaled by said subject after said administering indicates therapeutic efficacy.

13. The method in accordance to claim 12, wherein said diffuse parenchymal lung disease is caused by environmental factors.

14. The method in accordance to claim 12, wherein said diffuse parenchymal lung disease is a collagen vascular disease.

15. The method in accordance to claim 12, wherein said diffuse parenchymal lung disease is an idiopathic interstitial pneumonia.

16. The method in accordance to claim 12, wherein said diffuse parenchymal lung disease is sarcoidosis.

17. The method in accordance to claim 14, wherein said collagen vascular disease is scleroderma.

18. The method in accordance to claim 14, wherein said collagen vascular disease is rheumatoid arthritis.

19. The method in accordance to claim 15, wherein said idiopathic interstitial pneumonia is idiopathic pulmonary fibrosis.

20. The method in accordance to claim 15, wherein said idiopathic interstitial pneumonia is nonspecific interstitial pneumonia.

21. The method in accordance to claim 12, wherein said selective active-site mTOR inhibitor is a pyrazolopyrimidine.

22. The method in accordance to claim 12, wherein said selective active-site mTOR inhibitor is one of Torin 1, PP242, INK128, Ku-0063794 or WAY-600.

23. A method for inhibiting expression of α-SMA or collagen or fibronectin in a pulmonary fibroblast, comprising administering an amount of a selective active-site mTOR inhibitor to a pulmonary fibroblast in an subject suffering from a diffuse parenchymal lung disease in a therapeutically effective amount, wherein the administering is effective to inhibit expression of α-SMA or collagen or fibronectin in said pulmonary fibroblast.

24. The method in accordance to claim 23, wherein said diffuse parenchymal lung disease is caused by environmental factors.

25. The method in accordance to claim 23, wherein said diffuse parenchymal lung disease is a collagen vascular disease.

26. The method in accordance to claim 23, wherein said diffuse parenchymal lung disease is an idiopathic interstitial pneumonia.

27. The method in accordance to claim 23, wherein said diffuse parenchymal lung disease is sarcoidosis.

28. The method in accordance to claim 25, wherein said collagen vascular disease is scleroderma.

29. The method in accordance to claim 25, wherein said collagen vascular disease is rheumatoid arthritis.

30. The method in accordance to claim 26, wherein said idiopathic interstitial pneumonia is idiopathic pulmonary fibrosis.

31. The method in accordance to claim 26, wherein said idiopathic interstitial pneumonia is nonspecific interstitial pneumonia.

32. The method in accordance to claim 23, wherein said selective active-site mTOR inhibitor is a pyrazolopyrimidine.

33. The method of claim 23, wherein said selective active-site mTOR inhibitor is one of Torin 1, PP242, INK128, Ku-0063794 or WAY-600.


SEQUENCE LISTING (9 sequences)

SEQ ID NO: 1 - DNA, 20 bp, Homo sapiens
  primer_bind (1)..(20): sense primer for human PAI-1
tggaacaaggatgagatcag

SEQ ID NO: 2 - DNA, 20 bp, Homo sapiens
  primer_bind (1)..(20): antisense primer for human PAI-1
ccgttgaagtagagggcatt

SEQ ID NO: 3 - DNA, 20 bp, Homo sapiens
  primer_bind (1)..(20): sense primer for human alpha-SMA
ctgttccagccatccttcat

SEQ ID NO: 4 - DNA, 20 bp, Homo sapiens
  primer_bind (1)..(20): antisense primer for human alpha-SMA
ccgtgatctccttctgcatt

SEQ ID NO: 5 - DNA, 21 bp, Homo sapiens
  primer_bind (1)..(21): sense primer for glyceraldehyde-3-phosphate dehydrogenase
gaccccttcattgacctcaac

SEQ ID NO: 6 - DNA, 20 bp, Homo sapiens
  primer_bind (1)..(20): antisense primer for glyceraldehyde-3-phosphate dehydrogenase
cttctccatggtggtgaaga

SEQ ID NO: 7 - DNA, 21 bp, Artificial Sequence
  primer
cgggatgttcacaaccgaatt

SEQ ID NO: 8 - DNA, 21 bp, Artificial Sequence
  primer
aacaagaccttcgactcttcc

SEQ ID NO: 9 - DNA, 21 bp, Artificial Sequence
  primer
gttctccgaacgtgtcacgtt